(19) United States
(12) Patent Application Publication
(10) Pub. No.: US 2003/0220284 A1
(43) Pub. Date: Nov. 27, 2003

(54) Delivery of adenoviral DNA in a liposomal formulation for treatment of disease

(76) Inventors: Patricia Yotnda, Houston, TX (US); Alan R. Davis, Missouri City, TX (US); Nancy S. Templeton, Houston, TX (US); Malcolm K. Brenner, Bellaire, TX (US)

Correspondence Address: FULBRIGHT & JAWORSKI, LLP, 1301 MCKINNEY, SUITE 5100, HOUSTON, TX, 77010-3095, US

(21) Appl. No.: 10/370,949

(22) Filed: Feb. 21, 2003

Related U.S. Application Data
(60) Provisional application No. 60/359,205, filed on Feb. 22, 2002.
(60) Provisional application No. 60/383,246, filed on May 24, 2002.

(51) Int. Cl. A61K 48/00; A61K 9/127

(52) U.S. Cl. 514/44; 424/450

(57) ABSTRACT

The present invention is directed to methods and compositions regarding delivery of adenoviral DNA in a liposomal formulation for disease treatment. In specific embodiments, the adenoviral DNA is circular and the liposomal formulation is comprised of DOTAP. In other specific embodiments, the disease being treated is cancer, such as lung cancer.

The present invention claims priority to U.S. Provisional Patent Applications Serial No. 60/359,205, filed Feb. 22, 2002, and Ser. No. 60/383,246, filed May 24, 2002, both of which are incorporated by reference herein in their entirety.

### STATEMENT REGARDING FEDERALLY SPONSORED RESEARCH OR DEVELOPMENT

U.S. Government funds pursuant to NIH Grant No. CA78792 were used for the present invention. The U.S. Government may have certain rights in the invention.

### FIELD OF THE INVENTION

The present invention is related to the fields of molecular biology and cell biology. Specifically, the present invention regards methods and compositions directed to gene transfer for the treatment of disease. More specifically, it relates to a therapeutic adenoviral DNA complexed in a liposome for disease treatment.

### BACKGROUND OF THE INVENTION

Adenovector-mediated gene transfer is attractive because the vector can transduce many cell types (Caplen et al., 1995; Crystal et al., 1994; Lee et al., 1996; Bellon et al., 1997; Gahery-Segard et al., 1997; Tursz et al., 1996; Welsh et al., 1994; Zuckerman et al., 1999) with relative efficiency to produce substantial—albeit transient—levels of transgene expression (Zuantin et al., 1992; Wilson, 1993). However, adenoviral vectors (AV) also have several limitations, the most important of which is their marked immunogenicity (Brody et al., 1994; Jooss et al., 1998; Kaplan et al., 1996; Yan et al., 1995). The immune response to AV likely has three components. The first is the induction of pro-inflammatory cytokines such as IL6 and IL8, either as a result of direct exposure of monocytes and macrophages to adenovector coated proteins or to their activation by low levels of adenovector antigen expression on infected target cells. The second component is the induction of a humoral antibody response that neutralizes adenovirus before they reach their target cells (Gahery-Segard et al., 1997; Yang et al., 1995; Wohlfart, 1988; Gahery-Segard et al., 1997; Toogood et al., 1992). Even if such antibodies are absent initially (Kaplan and Smith, 1997), they may develop rapidly in humans following exposure to the vectors and preclude or severely handicap attempts at repeat administration of the vector as transgene expression wanes. The third is the cellular immune response targeted against the low level of adenovector antigens expressed by vector infected cells, which destroys these targets with consequent loss of transgene product (Brody et al., 1994; Kaplan et al., 1996; Yang et al., 1995; Jooss et al., 1998). The last of these problems may be addressed by deletion of additional regions in the adenovector genome such as E2A and E4 (Armentano et al., 1997), or by reintroduction of immunosuppressive adenovector genes (such as E3) (Ilan et al., 1997) or by complete elimination of all adenovector genes by generating a helper-dependent or gutless vector (O'Neal et al., 2000; Parks et al., 1999). However, it is uncertain whether these modifications will alter the acute inflammatory or humoral immune response problems.

One means of ensuring that Ad vectors escaped neutralization by the humoral immune response is to encapsulate them (Lee et al., 2000). Encapsulation fulfills a secondary function of modifying the targeting characteristics of adenovector so that a more restricted or entirely distinct population of cells is transduced (Fasbender et al., 1997). However, the liposomes used to date have been hindered by an inability to truly encapsulate the adenovector (Lee et al., 2000). Instead, they produce a “spaghetti and meatballs” appearance in which liposomal fragments of varying size and shape only partially coat and incompletely surround the particles that they nominally encapsulate (Templeton et al., 1997). Recently, an alternative type of liposome (1,2-dioleoyloxypropyl)-N,N,N-trimethylammonium chloride propane DOTAP:chol (cholesterol) has been described, which consists of a bilamellar liposomal envelope that can entirely surround the particles it contains (Templeton et al., 1997). Such bilamellar liposomes have efficiently delivered plasmid DNA to many tissues and organs, including lung and liver parenchyme (Templeton et al., 1997). The present invention addresses encapsulation of adenovectors and subsequent protection from the human humoral immune response, while retaining or increasing their known advantages of wide target cell range and high level gene expression in target cells in vitro and in vivo.

U.S. Pat. No. 6,133,243 is for liposomal-viral DNA complexes for treating disease. The invention relates to methods to treat cancer by administering to a tumor-bearing animal a liposomal adenoviral DNA complex, wherein the adenoviral DNA lacks a viral oncoprotein capable of binding to a functional p53 and that replicates and forms infectious virus in the tumor cells that lack functional p53.

U.S. Pat. No. 6,110,490 regards specific embodiments wherein there is a bi-or multi-layer membrane surrounding an internal aqueous liposome comprising at least one cationic lipopolyamine and at least one neutral lipid in a molar ratio range of about 0.02:1 to about 2.0:1, and the composition further comprises adenovirus particles. A specific example of lipid used for the liposome is DOTAP.

Lee et al. (2000) are directed to enhancement of adenoviral transduction with polycationic liposomes in vivo. Adenovirus harboring human placental alkaline phosphatase and lipofectamine of 1,3-di-oleoyloxy-2-(6-carboxyspermyl)-propylamide were utilized. An increased transfection efficiency was observed for the composition in CT26 tumor cell lines. In an animal model, the composition was distributed wider and deeper in a tumor mass. Adenoviral vectors included AdALP and Ad-mGM-CSF were utilized.

Sung et al. (2000) refers to cationic liposome-enhanced adenoviral gene transfer in a murine head and neck cancer model. The combination of DOSPER (Boehringer Mannheim; Indianapolis, Ind.) and an adenoviral vector (AdALP and AdmGM-CSF) had enhanced gene transfer both in vitro and in vivo compared to controls.

Buttgereit et al. (2000) describe efficient gene transfer into lymphoma cells using adenoviral vectors combined with liposomes. Transgene-expressing lymphoma cells were higher in number than those transfected with adenovirus alone. The recombinant adenoviral Ad-β-gal vector (E1- and E3-deleted replication-defective adenovirus type 5) carrying the E. coli lacZ gene encodes β-gal under control of the promoter of the Rous sarcoma virus.

U.S. Pat. No. 5,928,944, in specific embodiments, is directed to introducing a nucleic acid into a eukaryotic cell by contacting the cell with the nucleic acid, an adenovirus, and a cationic agent such as a liposome.

U.S. Pat. No. 5,908,635 refers to a liposome composition comprising a cationic lipopolyamine and a neutral lipid, wherein the cationic lipopolyamine comprises spermine-5-carboxy-glycinedioctadecylamide and, in specific embodiments, the composition comprises adenovirus.

U.S. Pat. No. 5,635,380 addresses a method of enhancing the delivery of a nucleic acid into a cell in vitro, comprising forming a complex comprising the nucleic acid linked via a cationic liposome to a virus and administering the complex to the cell, thereby enhancing the delivery of the nucleic acid into the cell.

The present invention being directed to liposomes comprising adenoviral DNA, particularly in circular form, overcomes deficiencies in the art, as described herein.

### BRIEF SUMMARY OF THE INVENTION

The present invention is directed to a system and method that facilitates gene transfer through the use of an adenoviral DNA complexed with a liposome.

In one embodiment of the present invention, there is a composition comprising an adenoviral DNA comprised in a liposome, wherein the adenoviral DNA is circular, and in other embodiments the composition is utilized for a therapeutic purpose. In one specific embodiment, the therapeutic purpose is the treatment of cancer. In a preferred embodiment, the adenoviral DNA is not housed within a viral particle, which helps to prevent eliciting an immune response from the individual to which it is administered.

The success of conditionally replicating oncolotic viruses (CROV) relies on biological differences between malignant and normal cells that allow viral replication only in the former. The most widely used of these CROV, adenovirus dl1520, was developed to take advantage of differences in p53 activity between normal and malignant cells. This E1b deletion mutant lacks the ability to bind p53 present in normal cells and fails to replicate. In tumor cells lacking a functional p53 pathway, viral replication and host cell destruction may readily occur. Although more recent data suggests that the selective lysis achieved by dl1520 may be independent of abnormalities associated with the p53 pathway, undoubtedly oncolotic responses have been obtained in pre-clinical and clinical studies. However, the approach has several limitations. Local injection of the virus is of no value for metastatic cancer, while systemic injection leads to high levels of uptake by hepatic cells and by vascular endothelium with potentially devastating consequences. Moreover, pre-existing or developing immune responses to adenoviral coat proteins may rapidly inactivate) the injected virus before sufficient uptake by malignant cells has occurred.

The present invention provides an approach comprising liposomal encapsulation of a circular adenoviral plasmid based on the dl1520 conditionally replicating oncolytic virus or the linear viral DNA of dl1520. The present inventors have previously described how such bilamellar liposomal encapsulation modifies the biodistribution of adenoviral vector and protects them from antibody neutralization, while permitting a high level of gene transfer to target cells. The present invention, however, comprises tumor uptake of a circular plasmid and linear viral DNA, which are then able to form infectious adenoviral particles within tumor cells. These in turn lead to tumor lysis in vitro and in vivo. These encapsulated conditionally replication-competent plasmids are a useful adjunction to conventional oncolytic vectors.

Thus, as described, adenoviral vectors have been widely used for gene therapy, but are limited both by the presence of a humoral immune response that dramatically decreases the level of transduction after re-injection, and by their requirement for target cells to express appropriate receptors such as CAR. To overcome both limits, in specific embodiments, adenovectors were encapsulated using bilamellar DOTAP:chol liposomes. Electron micrographs showed that these liposomes efficiently encapsulated the vectors, allowing CAR-independent adenovector transduction of otherwise resistant cells. DOTAP:chol encapsulated advectors encoding lac-Z or α1 antitrypsin inhibitor (AAT) were also functionally resistant ex vivo and in vivo to the neutralizing effects of human anti-adenoviral antibodies. Hence, bilamellar DOTAP:chol liposomes are useful for applications using adenovectors in which the target cells lack adenoviral receptors or in which the recipient already has or develops a neutralizing antibody response that would otherwise inactivate a re-administered vector.

Thus, the present invention is directed to the following embodiments:

In one embodiment of the present invention, there is a composition of matter, a liposome comprising adenoviral DNA, wherein the adenoviral DNA comprises a circular form and a therapeutic polynucleotide. In a specific embodiment, the adenoviral DNA comprises the therapeutic polynucleotide. In another specific embodiment, the adenoviral DNA is contained within the liposome. In a further specific embodiment, the adenoviral DNA is housed within an adenoviral particle. In additional specific embodiments, the adenoviral DNA is native adenoviral DNA, is recombinant adenoviral DNA, is replication-deficient, or is a combination thereof. In a specific embodiment, the adenoviral DNA is dl1520. In other specific embodiments, the liposome is a bilammelar liposome, is comprised of DOTAP, is comprised of extruded DOTAP-cholesterol, or a combination thereof. In a further specific embodiment, the composition further comprises humoral immune response-neutralizing activity.

In another embodiment of the present invention, there is a therapeutic composition, comprising a liposome, comprising adenoviral DNA, wherein the adenoviral DNA comprises a circular form; a therapeutic polynucleotide; and a pharmaceutical carrier. In an additional specific embodiment, the adenoviral DNA comprises the therapeutic polynucleotide. In an additional specific embodiment, the adenoviral DNA is contained within the liposome, the adenoviral DNA is native adenoviral DNA, the adenoviral DNA is recombinant, the adenoviral DNA is replication-deficient, or a combination thereof In a specific embodiment, the adenoviral DNA is dl1520. In other specific embodiments, the liposome is a bilammelar liposome, the liposome is comprised of DOTAP, the liposome is comprised of extruded DOTAP-cholesterol, or a combination thereof. In a specific embodiment, the composition further comprises humoral immune response-neutralizing activity. In one embodiment, there is a vaccine comprising a composition described herein.

In an additional embodiment of the present invention, there is a method of treating a disease in an individual comprising the step of administering to the individual a composition comprising a liposome, said liposome comprising adenoviral DNA, wherein the adenoviral DNA comprises a circular form; a therapeutic polynucleotide; and a pharmaceutical carrier. In a specific embodiment, the adenoviral DNA comprises the therapeutic polynucleotide, the adenoviral DNA is contained within the liposome, or a combination thereof. In an additional specific embodiment, the composition is administered at least a second time. In a specific embodiment, the individual is a warm-blooded animal, the animal is a human, the disease is cancer, the cancer is lung cancer, or a combination thereof. In other specific embodiments, the therapeutic polynucleotide encodes p53, BRCA1, BRCA2, a bone morphogenetic protein, an interleukin, thymidine kinase, or cytosine deaminase. In a specific embodiment, the adenoviral DNA is dl1520, the liposome is a bilammelar liposome, the liposome is comprised of DOTAP, the liposome is comprised of extruded DOTAP-cholesterol, or a combination thereof. In a specific embodiment, the composition further comprises humoral immune response-neutralizing activity.

In an additional embodiment of the present invention, there is a method of preventing a disease in an individual comprising the step of administering to the individual a composition comprising a liposome, said liposome comprising adenoviral DNA, wherein the adenoviral DNA comprises a circular form; a therapeutic polynucleotide; and a pharmaceutical carrier. In a specific embodiment, the composition is administered at least a second time.

In another embodiment of the present invention, there is a liposome comprising within a circular adenoviral DNA, wherein the adenoviral DNA comprises a therapeutic polynucleotide.

In another embodiment of the present invention, there is a liposome comprised of DOTAP, said liposome comprising adenoviral DNA, wherein the adenoviral DNA comprises a linear form; and a therapeutic polynucleotide. In a specific embodiment, the adenoviral DNA comprises said therapeutic polynucleotide, the adenoviral DNA is contained within the liposome, the adenoviral DNA is not housed within an adenoviral particle, or a combination thereof.

In an additional embodiment of the present invention, there is a mixture of liposomes and adenoviral DNA, wherein the adenoviral DNA is housed within the liposomes, said mixture substantially lacking adenoviral DNA outside said liposomes. In a specific embodiment, the adenoviral DNA comprises a therapeutic polynucleotide. In another specific embodiment, the adenoviral DNA is not housed within an adenoviral particle.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings, in which:

FIGS. 1A and 1B describe encapsulation of adenovirus. Adenovirus complexed to DOTAP:chol (FIG. 1A) and naked Adenovirus (FIG. 1B) were analyzed by Cryo-Electron Microscopy as described in Example 1. DOTAP was used at 4 mM or 20 mM with a 1/10 dilution of Ad-GFP virus stock (5×10¹² virus particles (vp)/ml, vp:1pfu=56).

FIG. 2 shows Ad-resistant cell line infection. CAR negative and positive cell lines were infected at 10³ vp per cell in the presence or absence of neutralizing serum. The cells were analyzed by FACS 24 hr-post infection. Results are expressed as the percentage of cells expressing GFP in 3 independent experiments.

FIG. 3 demonstrates that immune serum inhibits uncoated advectors at a higher dilution than liposomal virus. 293 cells were seeded in 96 well plates and infected with an Ad-lac-z virus either alone or following liposomal incorporation. Immune serum was added at dilutions ranging from 1/1 to 1/256. After 24 hrs incubation, the efficiency of infection was estimated in each well using a β-Galactosidase Enzyme Assay kit (Promega, Madison, Wis.). Absorbance was read at 410 nm with a spectrophotometer and report results as the percentage of the maximum absorbance obtained with virus in the absence of serum.

FIGS. 4A through 4L show the effects of re-administration of Ad-lac-z. Mice were injected at D0 and D30 with naked or liposome coated Ad-lac-z vector (10⁹ pfu/mice). Lungs and livers were harvested at the time points shown and analyzed for β-galactosidase expression. Livers of mice injected with virus alone harvested at day 7 (FIG. 4A), day 30 (FIG. 4B), day 60 from animals re-injected at day 30 (FIG. 4C). Lungs of the same animals were harvested: day 7 (FIG. 4D), 30 (FIG. 4E), and day 60 (FIG. 4F). Livers of mice injected with DOTAP:chol/Ad-lac-z were harvested at day 7 (FIG. 4G), day 30 (FIG. 4H), and 60 from mice re-injected at day 30 (FIG. 4I). Lungs of the same animals harvested: day 7 (FIG. 4J), 30 (FIG. 4K), and day 60 (FIG. 4L).

FIG. 5 shows re-administration of Ad-hAAT in the presence of neutralizing serum. Mice were injected IV in the tail vein with 10⁹ pfu of Ad-hAAT or Ad-hAAT/DOTAP:chol in the presence or absence of neutralizing serum. The level of hAAT produced was measured by ELISA one week after each injection. The mean values±standard errors are shown (n=5).

FIG. 6 demonstrates specific anti-Ad-hAAT in the serum. The level of anti-Ad-hAAT specific immunoglobulins present in the serum of injected mice (after receiving virus alone or coated with liposomes, in the presence or absence of neutralizing serum) was evaluated by ELISA after both the first and second injections. The mean values±standard errors are shown (n=5).

FIG. 7 shows an inflammatory response. Mice were injected IV in the tail vein with 10⁹ pfu of Ad-lac-z. One month after injection each mouse was re-injected with 2×10⁹ pfu. Serum of each mouse was harvested 6 hr and 24 hr after each injection and analyzed for IL6 and TNFα by ELISA. Results shown mean values±standard errors (n=5).

FIG. 8 illustrates an exemplary embodiment of circular adenoviral DNA construction.

FIGS. 9A through 9C depict efficiency of gene transfer using Ad5 plasmid/DOTAP (FIG. 9B) or adenovirus (FIG. 9C) compared to a control (FIG. 9A) in H1299 cells.

FIGS. 10A through 10D illustrate transfection of p53⁻ CAR⁺ and CAR^{low} cells by dl1520 virus. H1299 (p53 null, CAR+, human lung cancer) (FIGS. 10C and 10D) and T24 (p53 mutated (Ono y, Mol Urol. 2001 Spring;5(1):25-30), CAR^{low} human bladder cancer (van der Poel H G, J Urol July 2002;168(1):266-72) (FIGS. 10A and 10B) were infected with dl1520 at 1000 vp/cells. Plates were daily monitored for CPE and the assay was terminated at complete lysis of the monolayer. FIGS. 10A and 10C show non-transduced cells, and FIGS. 10B and 10D cells were transduced with virus. Only the H1299 cells (FIG. 10D) were sensitive to dl1520 treatment.

FIGS. 11A through 11B illustrate the cytopathic effect of dl1520 circular DNA on p53-cells (H1299). FIG. 11A illustrates differences between dl1520 circular DNA and linear viral dl1520 in the cytopathic effect assay. FIG. 11B is a higher magnification of H1299 cells lysis following circular dl1520 DNA transfection.

FIG. 12 shows production of viral particles with dl1520 DNA (Xho I digest).

FIGS. 13A and 13B illustrate the protective effect of DOTAP in the presence of neutralizing serum. FIG. 13A shows that 293 cells were incubated with an Ad5-Lac-z vector complexed with or without DOTAP and incubated with or without a neutralizing serum. The efficiency of infections in each well was estimated using a β-Galactosidase Enzyme Assay. H1299 cells were treated with dl1520 viruses (500 vp per cells) and linear/circular dl1520 plasmid complexed with DOTAP. Each condition was tested with or without a pre-incubation of 1 h with an Ad5 neutralizing serum. Plates were monitored daily for CPE until complete lysis of all of the groups. The inhibitory effect of the serum was evaluated by evaluating the time required for a complete cells lysis (FIG. 13B).

FIGS. 14A and 14B illustrate therapeutic effects of the circularized adenoviral DNA/liposome composition. FIG. 14A shows tumor growth inhibition with the composition following intratumoral injection into p53-null xenografted nude mice. FIG. 14B shows increased survival numbers in animals subjected to the circularized adenoviral DNA/liposome composition.

FIG. 15 shows tumor growth inhibition with the composition following intratumoral injection into p53-null xenografted SCID mice.

FIG. 16A shows inhibition of tumor growth with the composition following systemic injection into mice, wherein the composition is pre-treated with neutralizing serum. FIG. 16B shows immunocytochemistry to confirm that the virus replicates in the tumor.

### DETAILED DESCRIPTION OF THE INVENTION

I. Definitions

As used herein the specification, Via” or “an” may mean one or more. As used herein in the claim(s), when used in conjunction with the word “comprising”, the words “a” or “an” may mean one or more than one. As used herein “another” may mean at least a second or more.

The term “humoral immune response-neutralizing activity” as used herein refers to an activity that neutralizes, inhibits, protects from, impedes, or diminishes a humoral immune response. In a specific embodiment, the humoral immune response comprises an antibody response. A skilled artisan recognizes a humoral immune response pertains to antibodies dissolved in the blood or body fluids.

The term “liposome” as used herein refers to a closed structure comprising an outer lipid bi- or multi-layer membrane surrounding an internal aqueous space. Liposomes can be used to package any biologically active agent, such as an adenovirus, for delivery to cells. For example, DNA can be packaged into liposomes even in the case of adenoviral vectors of large size, which could potentially be maintained in a soluble form. Such liposome encapsulated DNA is ideally suited for direct application to in vivo systems by standard means, such as by a simple intravenous injection. In other embodiments, liposomes may entrap compounds varying in polarity and solubility in water and other solvents.

Liposomes are generally from a bilayer membrane in a uni- or multilamellar membranous structure. Generally, they may form hexagonal structures, and suspension of multilamellar vesicles. In order to form stable liposomes, the cationic lipopolyamine is combined with a neutral lipid. Such neutral lipids include triglycerides, diglycerides and cholesterol and are known in the art, for example as described in U.S. Pat. No. 5,438,044, which is incorporated herein by reference. In particular, a neutral phospholipid is preferred. In a specific embodiment, the liposome is a bilammellar DOTAP:cholesterol liposome. A skilled artisan recognizes that DOTAP is N-[1-(2,3-Dioleoyloxy) propyl]-N,N,N-trimethylammonium methyl-sulfate.

The term “native adenoviral DNA” as used herein refers to an adenoviral DNA that can be found in nature. For example, a polypeptide or polynucleotide sequence that is present in an organism (including viruses) that can be isolated from a source in nature and which has not been intentionally modified by man in the laboratory is native. In alternative embodiments, a recombinant adenoviral DNA is utilized. As used herein, the term “recombinant” indicates that a polynucleotide construct (e.g., an adenovirus genome) has been generated, in part, by intentional modification by man.

As used herein, the term “replication deficient virus” refers to a virus that preferentially inhibits cell proliferation or induces apoptosis in a predetermined cell population (e.g., cells substantially lacking p53 and/or RB function) that supports expression of a virus replication phenotype, and that is substantially unable to inhibit cell proliferation, induce apoptosis, or express a replication phenotype in cells comprising normal p53 and RB function levels characteristic of non-replicating, non-transformed cells. Typically, a replication deficient virus exhibits a substantial decrease in plaquing efficiency on cells comprising normal RB and/or p53 function.

The term “therapeutic polynucleotide” as used herein refers to a polynucleotide, such as a gene, which encodes a gene product that provides a therapeutic benefit to a recipient organism, said organism having a disease or deleterious medical condition.

II. The Present Invention

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

The present invention provides compositions and methods for treating an individual with a disease or medical condition. The composition comprises a liposome housing an adenoviral DNA, such as a circular adenoviral DNA, wherein the liposome also comprises a therapeutic polynucleotide for the treatment of said disease. In a specific embodiment, the circular adenoviral DNA further comprises the therapeutic polynucleotide. In another specific embodiment, the disease is cancer.

The present invention derives from two known embodiments. Templeton et al. (1997) demonstrate that a novel form of liposomes (extruded DOTAP:cholesterol) resulted in the DNA being contained on the interior of the liposome between two lipid bi-layers. This structure allows for the highly effective delivery of the DNA in vivo. In addition, it is known that a mutant of adenovirus (Barker and Berk, 1987), wherein the coding region of E1B 55 kd protein is deleted, preferentially replicates in and destroys tumor cells (Heise et al., 1997). This mutant, originally referred to as dl 520, is also referred to in the art as ONYX-015. ONYX-015 has shown great results in a number of pre-clinical (Heise et al., 1997; Shinoura et al., 1999) and clinical studies (Kim et al., 1998; Oncologist 5:432 (1999). However, ONYX-015 suffers from the same disadvantages of adenovirus vectors in general, including pre-existing and treatment-induced antibodies that neutralize ONYX-15. The current invention overcomes these limitations by delivering an adenoviral DNA, such as an adenovirus vector genome (e.g. ONYX-015), encapsulated in liposomes, such as extruded DOTAP-cholesterol liposomes.

In a specific embodiment of the present invention, the adenovirus DNA is comprised in the liposome in a circular form. Certain circular forms of adenovirus DNA that were infectious have been reported (Ruben et al., 1983; Graham, 1984). One, in fact (pFG140; Microbix, Inc.), encodes a fully infectious adenovirus. This DNA was successfully encapsulated in extruded DOTAP:cholesterol liposomes by the present inventors. The liposomes thus formed were found to be fully infectious on 293 cells, a cell line typically used for determining the injectivity of adenovirus. A skilled artisan recognizes that all adenovirus vectors, including ONYX-015, can be incorporated into the circular form.

In specific embodiments, the ONYX-015 adenovirus is used in compositions and/or methods of the present invention. Details regarding its manufacture and use are known in the art (Barker and Berk, 1987; Hasada and Berk, 1999, both incorporated by reference herein).

In a preferred embodiment of the present invention, the adenoviral DNA/liposome composition comprises the majority of adenoviral DNAs housed the liposomes. In prior art embodiments, the adenoviral DNA/liposome structures resemble a “spaghetti and meatballs” structure with a significant portion of the adenoviral DNA (the spaghetti) outside of the meatballs (the liposomes). The compositions of the present invention overcome this deficiency, as illustrated by electron microscopy. In specific embodiments, the adenoviral DNA/liposome composition substantially lacks adenoviral DNA outside the liposomes. In specific embodiments, this refers to at least about 80% of adenoviral DNA being within the liposomes, at least about 85%, at least about 90%, at least about 95%, at least about 97%, at least about 99%, or all.

Many human tumors have a functional deficiency in p53. Numerous studies have taken advantage of this phenomenon to use a conditionally replication competent adenovirus (Ad dl1520) that will grow in and lyse tumor cells while sparing normal tissues. However, success has been limited in part due to difficulties in reaching a sufficient high proportion of tumor cells. Pre-existing or developing immune response directed to viral proteins further decreases the efficacy of the approach. The present inventors have developed a liposome encapsulated conditionally replication competent plasmid based on dl1520 virus. Like the parent virus, this plasmid generates infectious particles following transfection of p53 defective but not p53 wt tumor cells or normal tissues. The anti-tumor efficacy of this infectious plasmid was demonstrated in mice with xenografted human tumors and was active both on local administration for subcutaneous tumors and intravenously for disseminated malignancy. Such liposomally encapsulated conditionally replication competent plasmids may complement the conventional strategy using viral particles in settings where liver uptake of adenoviral vector is undesirable and where the inhibitory effect of humoral responses on these vectors is problematic.

III. Liposomes and Lipid Compositions

The present invention in some embodiments utilizes lipid compositions, preferably liposomes.

Liposome transfection of viral DNA can be via liposomes comprised of, for example, phosphatidylcholine (PC), phosphatidylserine (PS), cholesterol (Chol), N-[1-(2,3-diolexyloxy) propyl]-N,N-trimethylammonium chloride (DOTMA), dioleoylphosphatidylethanolamine (DOPE), and/or 3β[N-(NN-dimethylaminoethane)-carbarmoyl cholesterol (DC-Chol), as well as other lipids known to those of skill in the art. Catatonic liposomes as described by Gao et al., Biochemical and Biophysical Research Communications, vol. 179: pages 280-285. (1991) are preferred in the instant invention. Gao et al. describes a novel cationic cholesterol derivative that can be synthesized in a single step. Liposomes made of this lipid are more efficient in transfection and less toxic to treated cells than those made with the reagent Lipofectin™.

Those of skill in the art will recognize that there are a variety of liposomal transfection techniques that will be useful in the present invention. Among these techniques are those described by Nicolau et al., Methods in Enzymology, vol. 149: pages 157-176 (1987), and liposomes comprised of DOTMA, such as those which are available commercially under the trademark Lipofectin™, from Vical, Inc. (San Diego, Calif.) may also be used.

Liposomes may be introduced into contact with cells to be transfected by a variety of methods. In cell culture, the liposomes are simply dispersed in the cell culture solution. However, for application in vivo liposomes are typically injected. The preferred method, as mentioned above, is direct injection into the tumor to limit immune rejection of the viral DNA. However, other modes of administration may be used. Intravenous injection allows liposome-mediated transfer of the viral DNA to target the liver and the spleen.

The lipid employed to make the liposomal complex can be any of the above-discussed lipids. In particular, DOTMA, DOPE, and/or DC-Chol may form all or part of the liposomal complex. In a preferred embodiment, the lipid will comprise DC-Chol and DOPE comprising a ratio of DC-Chol:DOPE between 1:20 and 20:1. More preferred are liposomes prepared from a ratio of DC-Chol:DOPE of about 1:10 to about 1:5.

As mentioned above, intravenously injected liposomes are taken up essentially in the liver and the spleen by the macrophages of the reticulendothelial system. The specific site of uptake of injected liposomes appears to be mainly spleen macrophages and liver Kupffer cells. Intravenous injection of liposomes/DNA complexes can lead to the uptake of DNA by these cellular sites, and result in the expression of a gene product encoded in the DNA (Nicolau, Biol. Cell, vol. 47: pages 121-130 (1983). Thus, liposomal viral DNA complexes of the inventions can effectively be targeted to tumors of the liver and/or spleen that originate in these regions, or to tumors that originate elsewhere and metastasize to these organs.

Intravenous injection is one means of realizing site-specific delivery of the liposome encapsulated viral DNA sequences. Such can be delivered selectively to the appropriate target tumor cells by other means, and a preferred means is via a catheter, as described by Nabel et al., Science, vol. 249: pages 1285-1288 (1990). For example, Nabel et al., above, teach injection via a catheter into the arterial wall. Importantly, these methods permit delivering of the liposome viral DNA sequences at a specific site in vivo, and not just to the liver and spleen cells which are accessible via intravenous injection.

Although the preferred embodiment of the present invention utilizes lipids in the form of liposomes, in certain embodiments the present invention concerns a novel composition comprising one or more lipids associated with at least one adenovirus, wherein the adenovirus preferably comprises a therapeutic polynucleotide. A lipid is a substance that is characteristically insoluble in water and extractable with an organic solvent. Lipids include, for example, the substances comprising the fatty droplets that naturally occur in the cytoplasm as well as the class of compounds which are well known to those of skill in the art which contain long-chain aliphatic hydrocarbons and their derivatives, such as fatty acids, alcohols, amines, amino alcohols, and aldehydes. Of course, compounds other than those specifically described herein that are understood by one of skill in the art as lipids are also encompassed by the compositions and methods of the present invention.

A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof.

A. Lipid Types

A neutral fat may comprise a glycerol and a fatty acid. A typical glycerol is a three carbon alcohol. A fatty acid generally is a molecule comprising a carbon chain with an acidic moeity (e.g., carboxylic acid) at an end of the chain. The carbon chain may of a fatty acid may be of any length, however, it is preferred that the length of the carbon chain be of from about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, to about 30 or more carbon atoms, and any range derivable therein. However, a preferred range is from about 14 to about 24 carbon atoms in the chain portion of the fatty acid, with about 16 to about 18 carbon atoms being particularly preferred in certain embodiments. In certain embodiments the fatty acid carbon chain may comprise an odd number of carbon atoms, however, an even number of carbon atoms in the chain may be preferred in certain embodiments. A fatty acid comprising only single bonds in its carbon chain is called saturated, while a fatty acid comprising at least one double bond in its chain is called unsaturated.

Specific fatty acids include, but are not limited to, linoleic acid, oleic acid, palmitic acid, linolenic acid, stearic acid, lauric acid, myristic acid, arachidic acid, palmitoleic acid, arachidonic acid ricinoleic acid, tuberculosteric acid, lactobacillic acid. An acidic group of one or more fatty acids is covalently bonded to one or more hydroxyl groups of a glycerol. Thus, a monoglyceride comprises a glycerol and one fatty acid, a diglyceride comprises a glycerol and two fatty acids, and a triglyceride comprises a glycerol and three fatty acids.

A phospholipid generally comprises either glycerol or an sphingosine moiety, an ionic phosphate group to produce an amphipathic compound, and one or more fatty acids. Types of phospholipids include, for example, phosphoglycerides, wherein a phosphate group is linked to the first carbon of glycerol of a diglyceride, and sphingophospholipids (e.g., sphingomyelin), wherein a phosphate group is esterified to a sphingosine amino alcohol. Another example of a sphingophospholipid is a sulfatide, which comprises an ionic sulfate group that makes the molecule amphipathic. A phospholipid may, of course, comprise further chemical groups, such as for example, an alcohol attached to the phosphate group. Examples of such alcohol groups include serine, ethanolamine, choline, glycerol and inositol. Thus, specific phosphoglycerides include a phosphatidyl serine, a phosphatidyl ethanolamine, a phosphatidyl choline, a phosphatidyl glycerol or a phosphotidyl inositol. Other phospholipids include a phosphatidic acid or a diacetyl phosphate. In one aspect, a phosphatidylcholine comprises a dioleoylphosphatidylcholine (a.k.a. cardiolipin), an egg phosphatidylcholine, a dipalmitoyl phosphalidycholine, a monomyristoyl phosphatidylcholine, a monopalmitoyl phosphatidylcholine, a monostearoyl phosphatidylcholine, a monooleoyl phosphatidylcholine, a dibutroyl phosphatidylcholine, a divaleroyl phosphatidylcholine, a dicaproyl phosphatidylcholine, a diheptanoyl phosphatidylcholine, a dicapryloyl phosphatidylcholine or a distearoyl phosphatidylcholine.

A glycolipid is related to a sphinogophospholipid, but comprises a carbohydrate group rather than a phosphate group attached to a primary hydroxyl group of the sphingosine. A type of glycolipid called a cerebroside comprises one sugar group (e.g., a glucose or galactose) attached to the primary hydroxyl group. Another example of a glycolipid is a ganglioside (e.g., a monosialoganglioside, a GM1), which comprises about 2, about 3, about 4, about 5, about 6, to about 7 or so sugar groups, that may be in a branched chain, attached to the primary hydroxyl group. In other embodiments, the glycolipid is a ceramide (e.g., lactosylceramide).

A steroid is a four-membered ring system derivative of a phenanthrene. Steroids often possess regulatory functions in cells, tissues and organisms, and include, for example, hormones and related compounds in the progestagen (e.g., progesterone), glucocoricoid (e.g., cortisol), mineralocorticoid (e.g., aldosterone), androgen (e.g., testosterone) and estrogen (e.g., estrone) families. Cholesterol is another example of a steroid, and generally serves structural rather than regulatory functions. Vitamin D is another example of a sterol, and is involved in calcium absorption from the intestine.

A terpene is a lipid comprising one or more five carbon isoprene groups. Terpenes have various biological functions, and include, for example, vitamin A, coenzyme Q and carotenoids (e.g., lycopene and β-carotene).

B. Charged and Neutral Lipid Compositions

In certain embodiments, a lipid component of a composition is uncharged or primarily uncharged. In one embodiment, a lipid component of a composition comprises one or more neutral lipids. In another aspect, a lipid component of a composition may be substantially free of anionic and cationic lipids, such as certain phospholipids (e.g., phosphatidyl choline) and cholesterol. In certain aspects, a lipid component of an uncharged or primarily uncharged lipid composition comprises about 95%, about 96%, about 97%, about 98%, about 99% or 100% lipids without a charge, substantially uncharged lipid(s), and/or a lipid mixture with equal numbers of positive and negative charges.

In other aspects, a lipid composition may be charged. For example, charged phospholipids may be used for preparing a lipid composition according to the present invention and can carry a net positive charge or a net negative charge. In a non-limiting example, diacetyl phosphate can be employed to confer a negative charge on the lipid composition, and stearylamine can be used to confer a positive charge on the lipid composition.

C. Making Lipids

Lipids can be obtained from natural sources, commercial sources or chemically synthesized, as would be known to one of ordinary skill in the art. For example, phospholipids can be from natural sources, such as egg or soybean phosphatidylcholine, brain phosphatidic acid, brain or plant phosphatidylinositol, heart cardiolipin and plant or bacterial phosphatidylethanolamine. In another example, lipids suitable for use according to the present invention can be obtained from commercial sources. For example, dimyristyl phosphatidylcholine (“DMPC”) can be obtained from Sigma Chemical Co., dicetyl phosphate (“DCP”) is obtained from K & K Laboratories (Plainview, N.Y.); cholesterol (“Chol”) is obtained from Calbiochem-Behring; dimyristyl phosphatidylglycerol (“DMPG”) and other lipids may be obtained from Avanti Polar Lipids, Inc. (Birmingham, Ala.). In certain embodiments, stock solutions of lipids in chloroform or chloroform/methanol can be stored at about −20° C. Preferably, chloroform is used as the only solvent since it is more readily evaporated than methanol.

D. Lipid Composition Structures

In a preferred embodiment of the invention, the adenovirus is associated with a lipid. An adenovirus associated with a lipid may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure. A lipid or lipid/adenovirus associated composition of the present invention is not limited to any particular structure. For example, they may also simply be interspersed in a solution, possibly forming aggregates which are not uniform in either size or shape. In another example, they may be present in a bilayer structure, as micelles, or with a “collapsed” structure. In another non-limiting example, a lipofectamine(Gibco BRL)-adenovirus or Superfect (Qiagen)-adenovirus complex is also contemplated.

In certain embodiments, a lipid composition may comprise about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or any range derivable therein, of a particular lipid, lipid type or non-lipid component such as a drug, protein, sugar, nucleic acids or other material disclosed herein or as would be known to one of skill in the art. In a non-limiting example, a lipid composition may comprise about 10% to about 20% neutral lipids, and about 33% to about 34% of a cerebroside, and about 1% cholesterol. In another non-limiting example, a liposome may comprise about 4% to about 12% terpenes, wherein about 1% of the micelle is specifically lycopene, leaving about 3% to about 11% of the liposome as comprising other terpenes; and about 10% to about 35% phosphatidyl choline, and about 1% of a drug. Thus, it is contemplated that lipid compositions of the present invention may comprise any of the lipids, lipid types or other components in any combination or percentage range.

1. Emulsions

A lipid may be comprised in an emulsion. A lipid emulsion is a substantially permanent heterogenous liquid mixture of two or more liquids that do not normally dissolve in each other, by mechanical agitation or by small amounts of additional substances known as emulsifiers. Methods for preparing lipid emulsions and adding additional components are well known in the art (e.g., Modem Pharmaceutics, 1990, incorporated herein by reference).

For example, one or more lipids are added to ethanol or chloroform or any other suitable organic solvent and agitated by hand or mechanical techniques. The solvent is then evaporated from the mixture leaving a dried glaze of lipid. The lipids are resuspended in aqueous media, such as phosphate buffered saline, resulting in an emulsion. To achieve a more homogeneous size distribution of the emulsified lipids, the mixture may be sonicated using conventional sonication techniques, further emulsified using microfluidization (using, for example, a Microfluidizer, Newton, Mass.), and/or extruded under high pressure (such as, for example, 600 psi) using an Extruder Device (Lipex Biomembranes, Vancouver, Canada).

2. Micelles

A lipid may be comprised in a micelle. A micelle is a cluster or aggregate of lipid compounds, generally in the form of a lipid monolayer, and may be prepared using any micelle producing protocol known to those of skill in the art (e.g., Canfield et al., 1990; El-Gorab et al, 1973; Colloidal Surfactant, 1963; and Catalysis in Micellar and Macromolecular Systems, 1975, each incorporated herein by reference). For example, one or more lipids are typically made into a suspension in an organic solvent, the solvent is evaporated, the lipid is resuspended in an aqueous medium, sonicated and then centrifuged.

E. Liposomes

As indicated above, in preferred embodiments a lipid comprises a liposome. A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition.

A multilamellar liposome has multiple lipid layers separated by aqueous medium. They form spontaneously when lipids comprising phospholipids are suspended in an excess of aqueous solution. The lipid components undergo self-rearrangement before the formation of closed structures and entrap water and dissolved solutes between the lipid bilayers (Ghosh and Bachhawat, 1991). Lipophilic molecules or molecules with lipophilic regions may also dissolve in or associate with the lipid bilayer.

In certain less preferred embodiments, phospholipids from natural sources, such as egg or soybean phosphatidylcholine, brain phosphatidic acid, brain or plant phosphatidylinositol, heart cardiolipin and plant or bacterial phosphatidylethanolamine are preferably not used as the primary phosphatide, i.e., constituting 50% or more of the total phosphatide composition or a liposome, because of the instability and leakiness of the resulting liposomes.

In particular embodiments, a lipid and/or adenovirus may be, for example, encapsulated in the aqueous interior of a liposome, interspersed within the lipid bilayer of a liposome, attached to a liposome via a linking molecule that is associated with both the liposome and the adenovirus, entrapped in a liposome, complexed with a liposome, etc.

1. Making Liposomes

A liposome used according to the present invention can be made by different methods, as would be known to one of ordinary skill in the art. Phospholipids can form a variety of structures other than liposomes when dispersed in water, depending on the molar ratio of lipid to water. At low ratios the lipolsome is the preferred structure.

For example, a phospholipid (Avanti Polar Lipids, Alabaster, Ala.), such as for example the neutral phospholipid dioleoylphosphatidylcholine (DOPC), is dissolved in tert-butanol. The lipid(s) is then mixed with the adenovirus, and/or other component(s). Tween 20 is added to the lipid mixture such that Tween 20 is about 5% of the composition's weight. Excess tert-butanol is added to this mixture such that the volume of tert-butanol is at least 95%. The mixture is vortexed, frozen in a dry ice/acetone bath and lyophilized overnight. The lyophilized preparation is stored at −20° C. and can be used up to three months. When required the lyophilized liposomes are reconstituted in 0.9% saline. The average diameter of the particles obtained using Tween 20 for encapsulating the adenovirus is about 0.7 to about 1.0 μm in diameter.

Alternatively, a liposome can be prepared by mixing lipids in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min. to 2 hours, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

In other alternative methods, liposomes can be prepared in accordance with other known laboratory procedures (e.g., see Bangham et al, 1965; Gregoriadis, 1979; Deamer and Uster 1983, Szoka and Papahadjopoulos, 1978, each incorporated herein by reference in relevant part). These methods differ in their respective abilities to entrap aqueous material and their respective aqueous space-to-lipid ratios.

The dried lipids or lyophilized liposomes prepared as described above may be dehydrated and reconstituted in a solution of inhibitory peptide and diluted to an appropriate concentration with an suitable solvent, e.g., DPBS. The mixture is then vigorously shaken in a vortex mixer. Unencapsulated additional materials, such as agents including but not limited to hormones, drugs, nucleic acid constructs and the like, are removed by centrifugation at 29,000×g and the liposomal pellets washed. The washed liposomes are resuspended at an appropriate total phospholipid concentration, e.g., about 50-200 mM. The amount of additional material or active agent encapsulated can be determined in accordance with standard methods. After determination of the amount of additional material or active agent encapsulated in the liposome preparation, the liposomes may be diluted to appropriate concentrations and stored at 4° C. until use. A pharmaceutical composition comprising the liposomes will usually include a sterile, pharmaceutically acceptable carrier or diluent, such as water or saline solution.

The size of a liposome varies depending on the method of synthesis. Liposomes in the present invention can be a variety of sizes. In certain embodiments, the liposomes are small, e.g., less than about 100 nm, about 90 nm, about 80 nm, about 70 nm, about 60 nm, or less than about 50 nm in external diameter. In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; International Applications PCT/US85/01161 and PCT/US89/05040; U.K. Patent Application GB 2193095 A; Mayer et al., 1986; Hope et al., 1985; Mayhew et al. 1987; Mayhew et al., 1984; Cheng et al., 1987; and Liposome Technology, 1984, each incorporated herein by reference).

A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules may form a bilayer, known as a lamella, of the arrangement XY-YX. Aggregates of lipids may form when the hydrophilic and hydrophobic parts of more than one lipid molecule become associated with each other. The size and shape of these aggregates will depend upon many different variables, such as the nature of the solvent and the presence of other compounds in the solution.

The production of lipid formulations often is accomplished by sonication or serial extrusion of liposomal mixtures after (I) reverse phase evaporation (II) dehydration-rehydration (III) detergent dialysis and (IV) thin film hydration. In one aspect, a contemplated method for preparing liposomes in certain embodiments is heating sonicating, and sequential extrusion of the lipids through filters or membranes of decreasing pore size, thereby resulting in the formation of small, stable liposome structures. This preparation produces liposomal/adenovirus or liposomes only of appropriate and uniform size, which are structurally stable and produce maximal activity. Such techniques are well-known to those of skill in the art (see, for example Martin, 1990).

Once manufactured, lipid structures can be used to encapsulate compounds that are toxic (e.g., chemotherapeutics) or labile (e.g., nucleic acids) when in circulation. The physical characteristics of liposomes depend on pH, ionic strength and/or the presence of divalent cations. Liposomes can show low permeability to ionic and/or polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and/or results in an increase in permeability to ions, sugars and/or drugs. Liposomal encapsulation has resulted in a lower toxicity and a longer serum half-life for such compounds (Gabizon et al., 1990).

Liposomes interact with cells to deliver agents via four different mechanisms: Endocytosis by phagocytic cells of the reticuloendothelial system such as macrophages and/or neutrophils; adsorption to the cell surface, either by nonspecific weak hydrophobic and/or electrostatic forces, and/or by specific interactions with cell-surface components; fusion with the plasma cell membrane by insertion of the lipid bilayer of the liposome into the plasma membrane, with simultaneous release of liposomal contents into the cytoplasm; and/or by transfer of liposomal lipids to cellular and/or subcellular membranes, and/or vice versa, without any association of the liposome contents. Varying the liposome formulation can alter which mechanism is operative, although more than one may operate at the same time.

Numerous disease treatments are using lipid based gene transfer strategies to enhance conventional or establish novel therapies, in particular therapies for treating hyperproliferative diseases. Advances in liposome formulations have improved the efficiency of gene transfer in vivo (Templeton et al., 1997; U.S. Pat. No. 6,413,544) and it is contemplated that liposomes are prepared by these methods. Alternate methods of preparing lipid-based formulations for nucleic acid delivery are described (W0 99/18933).

In another liposome formulation, an amphipathic vehicle called a solvent dilution microcarrier (SDMC) enables integration of particular molecules into the bi-layer of the lipid vehicle (U.S. Pat. No. 5,879,703). The SDMCs can be used to deliver lipopolysaccharides, polypeptides, nucleic acids and the like. Of course, any other methods of liposome preparation can be used by the skilled artisan to obtain a desired liposome formulation in the present invention.

2. Liposome Targeting

Association of the adenovirus with a liposome may improve biodistribution and other properties of the adenovirus. For example, liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et a., 1980). Successful liposome-mediated gene transfer in rats after intravenous injection has also been accomplished (Nicolau et al., 1987).

It is contemplated that a liposome/adenovirus composition may comprise additional materials for delivery to a tissue. For example, in certain embodiments of the invention, the lipid or liposome may be associated with a hemagglutinating virus (HVJ). This has been shown to facilitate fusion with the cell membrane and promote cell entry of liposome-encapsulated DNA (Kaneda et al., 1989). In another example, the lipid or liposome may be complexed or employed in conjunction with nuclear non-histone chromosomal proteins (HMG-1) (Kato et al., 1991). In yet further embodiments, the lipid may be complexed or employed in conjunction with both HVJ and HMG-1.

Targeted delivery is achieved by the addition of ligands without compromising the ability of these liposomes deliver large amounts of adenovirus. It is contemplated that this will enable delivery to specific cells, tissues and organs. The targeting specificity of the ligand-based delivery systems are based on the distribution of the ligand receptors on different cell types. The targeting ligand may either be non-covalently or covalently associated with the lipid complex, and can be conjugated to the liposomes by a variety of methods.

a. Cross-Linkers

Bifunctional cross-linking reagents have been extensively used for a variety of purposes including preparation of affinity matrices, modification and stabilization of diverse structures, identification of ligand and receptor binding sites, and structural studies. Homobifunctional reagents that carry two identical functional groups proved to be highly efficient in inducing cross-linking between identical and different macromolecules or subunits of a macromolecule, and linking of polypeptide ligands to their specific binding sites. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, sulfhydryl, guanidino, indole, carboxyl specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group.

Exemplary methods for cross-linking ligands to liposomes are described in U.S. Pat. No. 5,603,872 and U.S. Pat. No. 5,401,511, each specifically incorporated herein by reference in its entirety). Various ligands can be covalently bound to liposomal surfaces through the cross-linking of amine residues. Liposomes, in particular, multilamellar vesicles (MLV) or unilamellar vesicles such as microemulsified liposomes (MEL) and large unilamellar liposomes (LUVET), each containing phosphatidylethanolamine (PE), have been prepared by established procedures. The inclusion of PE in the liposome provides an active functional residue, a primary amine, on the liposomal surface for cross-linking purposes. Ligands such as epidermal growth factor (EGF) have been successfully linked with PE-liposomes. Ligands are bound covalently to discrete sites on the liposome surfaces. The number and surface density of these sites will be dictated by the liposome formulation and the liposome type. The liposomal surfaces may also have sites for non-covalent association. To form covalent conjugates of ligands and liposomes, cross-linking reagents have been studied for effectiveness and biocompatibility. Cross-linking reagents include glutaraldehyde (GAD), bifunctional oxirane (OXR), ethylene glycol diglycidyl ether (EGDE), and a water soluble carbodiimide, preferably 1-ethyl-3-(3-dimethylaminopropyl) carbodiimide (EDC). Through the complex chemistry of cross-linking, linkage of the amine residues of the recognizing substance and liposomes is established.

In another example, heterobifunctional cross-linking reagents and methods of using the cross-linking reagents are described (U.S. Pat. No. 5,889,155, specifically incorporated herein by reference in its entirety). The cross-linking reagents combine a nucleophilic hydrazide residue with an electrophilic maleimide residue, allowing coupling in one example, of aldehydes to free thiols. The cross-linking reagent can be modified to cross-link various functional groups and is thus useful for cross-linking polypeptides and sugars. Table 1 details certain hetero-bifunctional cross-linkers considered useful in the present invention.

TABLE 1
HETERO-BIFUNCTIONAL CROSS-LINKERS

| | | | Spacer Arm |
|---|---|---|---|
| | | | Length\after |
| | | | cross- |
| Linker | Reactive Toward | Advantages and Applications | linking |
| SMPT | Primary amines | Greater stability | 11.2 A |
| | Sulfhydryls | | |
| SPDP | Primary amines | Thiolation | 6.8 A |
| | Sulfhydryls | Cleavable cross-linking | |
| LC-SPDP | Primary amines | Extended spacer arm | 15.6 A |
| | Sulfhydryls | | |
| Sulfo-LC-SPDP | Primary amines | Extended spacer arm | 15.6 A |
| | Sulfhydryls | Water-soluble | |
| SMCC | Primary amines | Stable maleimide reactive group | 11.6 A |
| | Sulfhydryls | Enzyme-antibody conjugation | |
| | | Hapten-carrier protein conjugation | |
| Sulfo- | Primary amines | Stable maleimide reactive group | 11.6 A |
| SMCC | Sulfhydryls | Water-soluble | |
| | | Enzyme-antibody conjugation | |
| MBS | Primary amines | Enzyme-antibody conjugation | 9.9 A |
| | Sulfhydryls | Hapten-carrier protein conjugation | |
| Sulfo-MBS | Primary amines | Water-soluble | 9.9 A |
| | Sulfhydryls | | |
| SIAB | Primary amines | Enzyme-antibody conjugation | 10.6 A |
| | Sulfhydryls | | |
| Sulfo-SIAB | Primary amines | Water-soluble | 10.6 A |
| | Sulfhydryls | | |
| SMPB | Primary amines | Extended spacer arm | 14.5 A |
| | Sulfhydryls | Enzyme-antibody conjugation | |
| Sulfo- | Primary amines | Extended spacer arm | 14.5 A |
| SMPB | Sulfhydryls | Water-soluble | |
| EDC/Sulfo- | Primary amines | Hapten-Carrier conjugation | 0 |
| NHS | Carboxyl groups | | |
| ABH | Carbohydrates | Reacts with sugar groups | 11.9 A |
| | Nonselective | | |

In instances where a particular polypeptide does not contain a residue amenable for a given cross-linking reagent in its native sequence, conservative genetic or synthetic amino acid changes in the primary sequence can be utilized.

b. Targeting Ligands

The targeting ligand can be either anchored in the hydrophobic portion of the complex or attached to reactive terminal groups of the hydrophilic portion of the complex. The targeting ligand can be attached to the liposome via a linkage to a reactive group, e.g., on the distal end of the hydrophilic polymer. Preferred reactive groups include amino groups, carboxylic groups, hydrazide groups, and thiol groups. The coupling of the targeting ligand to the hydrophilic polymer can be performed by standard methods of organic chemistry that are known to those skilled in the art. In certain embodiments, the total concentration of the targeting ligand can be from about 0.01 to about 10% mol.

Targeting ligands are any ligand specific for a characteristic component of the targeted region. Preferred targeting ligands include proteins such as polyclonal or monoclonal antibodies, antibody fragments, or chimeric antibodies, enzymes, or hormones, or sugars such as mono-, oligo- and poly-saccharides (see, Heath et al., Chem. Phys. Lipids 40:347 (1986)) For example, disialoganglioside GD2 is a tumor antigen that has been identified neuroectodermal origin tumors, such as neuroblastoma, melanoma, small-cell lung carcenoma, glioma and certain sarcomas (Mujoo et al., 1986, Schulz et al., 1984). Liposomes containing anti-disialoganglioside GD2 monoclonal antibodies have been used to aid the targeting of the liposomes to cells expressing the tumor antigen (Montaldo et al., 1999; Pagan et al., 1999). In another non-limiting example, breast and gynecological cancer antigen specific antibodies are described in U.S. Pat. No. 5,939,277, incorporated herein by reference. In a further non-limiting example, prostate cancer specific antibodies are disclosed in U.S. Pat. No. 6,107,090, incorporated herein by reference. Thus, it is contemplated that the antibodies described herein or as would be known to one of ordinary skill in the art may be used to target specific tissues and cell types in combination with the compositions and methods of the present invention. In certain embodiments of the invention, contemplated targeting ligands interact with integrins, proteoglycans, glycoproteins, receptors or transporters. Suitable ligands include any that are specific for cells of the target organ, or for structures of the target organ exposed to the circulation as a result of local pathology, such as tumors.

In certain embodiments of the present invention, in order to enhance the transduction of cells, to increase transduction of target cells, or to limit transduction of undesired cells, antibody or cyclic peptide targeting moieties (ligands) are associated with the lipid complex. Such methods are known in the art. For example, liposomes have been described further that specifically target cells of the mammalian central nervous system (U.S. Pat. No. 5,786,214, incorporated herein by reference). The liposomes are composed essentially of N-glutarylphosphatidylethanolamine, cholesterol and oleic acid, wherein a monoclonal antibody specific for neuroglia is conjugated to the liposomes. It is contemplated that a monoclonal antibody or antibody fragment may be used to target delivery to specific cells, tissues, or organs in the animal, such as for example, brain, heart, lung, liver, etc.

Still further, an adenovirus may be delivered to a target cell via receptor-mediated delivery and/or targeting vehicles comprising a lipid or liposome. These take advantage of the selective uptake of macromolecules by receptor-mediated endocytosis that will be occurring in a target cell. In view of the cell type-specific distribution of various receptors, this delivery method adds another degree of specificity to the present invention.

Thus, in certain aspects of the present invention, a ligand will be chosen to correspond to a receptor specifically expressed on the target cell population. A cell-specific adenovirus delivery and/or targeting vehicle may comprise a specific binding ligand in combination with a liposome. The adenoviruses to be delivered are housed within a liposome and the specific binding ligand is functionally incorporated into a liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In certain embodiments, a receptor-mediated delivery and/or targeting vehicles comprise a cell receptor-specific ligand and an adenovirus-binding agent. Others comprise a cell receptor-specific ligand to which adenovirus to be delivered has been operatively attached. For example, several ligands have been used for receptor-mediated gene transfer (Wu and Wu, 1987; Wagner et al., 1990; Perales et al., 1994; Myers, EPO 0273085), which establishes the operability of the technique. In another example, specific delivery in the context of another mammalian cell type has been described (Wu and Wu, 1993; incorporated herein by reference).

In still further embodiments, the specific binding ligand may comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolauet al., 1987). The asialoglycoprotein, asialofetuin, which contains terminal galactosyl residues, also has been demonstrated to target liposomes to the liver (Spanjer and Scherphof, 1983; Hara et al., 1996). The sugars mannosyl, fucosyl or N-acetyl glucosamine, when coupled to the backbone of a polypeptide, bind the high affinity manose receptor (U.S. Pat. No. 5,432,260, specifically incorporated herein by reference in its entirety). It is contemplated that the cell or tissue-specific transforming constructs of the present invention can be specifically delivered into a target cell or tissue in a similar manner.

In another example, lactosyl ceramide, and peptides that target the LDL receptor related proteins, such as apolipoprotein E3 (“Apo E”) have been useful in targeting liposomes to the liver (Spanjer and Scherphof, 1983; WO 98/0748).

Folate and the folate receptor have also been described as useful for cellular targeting (U.S. Pat. No. 5,871,727). In this example, the vitamin folate is coupled to the complex. The folate receptor has high affinity for its ligand and is overexpressed on the surface of several malignant cell lines, including lung, breast and brain tumors. Anti-folate such as methotrexate may also be used as targeting ligands. Transferrin mediated delivery systems target a wide range of replicating cells that express the transferrin receptor (Gilliland et al., 1980).

c. Liposome/Nucleic Acid Combinations

In certain embodiments, a liposome/adenovirus may comprise a therapeutic nucleic acid, such as, for example, an oligonucleotide, a polynucleotide or a nucleic acid construct (e.g., an expression vector). The polynucleotide comprising the therapeutic nucleic acid preferably comprises appropriate at least one regulatory element, which are well known in the art, such as a promoter. In one embodiment the regulatory element operates under the conditions of the cell in which the composition comprising the polynucleotide is targeted, such as a cancer cell. Where a bacterial promoter is employed in the DNA construct that is to be transfected into eukaryotic cells, it also will be desirable to include within the liposome an appropriate bacterial polymerase.

It is contemplated that when the liposome/adenovirus composition comprises a cell or tissue specific nucleic acid, this technique may have applicability in the present invention. In certain embodiments, lipid-based non-viral formulations provide an alternative to viral gene therapies. Although many cell culture studies have documented lipid-based non-viral gene transfer, systemic gene delivery via lipid-based formulations has been limited. Although a skilled artisan recognizes that numerous studies show liposomes alone are non-toxic, in some embodiments toxicity occurs when liposomes are mixed with high amounts of plasmid DNA and administered. In some embodiments, a limitation of non-viral lipid-based gene delivery is the toxicity of the cationic lipids that comprise the non-viral delivery vehicle. The in vivo toxicity of liposomes partially explains the discrepancy between in vitro and in vivo gene transfer results. Another factor contributing to this contradictory data is the difference in liposome stability in the presence and absence of serum proteins. The interaction between liposomes and serum proteins has a dramatic impact on the stability characteristics of liposomes (Yang and Huang, 1997). Cationic liposomes attract and bind negatively charged serum proteins. In some embodiments, liposomes coated by serum proteins are either dissolved or taken up by macrophages leading to their removal from circulation, although it is known that improved formulations of liposomes mixed with nucleic acids are stable at high concentrations of serum that match physiological concentrations in the serum (Smyth Templeton, 2003). Current in vivo liposomal delivery methods use aerosolization, subcutaneous, intradermal, intratumoral, or intracranial injection to avoid the toxicity and stability problems associated with cationic lipids in the circulation. The interaction of liposomes and plasma proteins is largely responsible for the disparity between the efficiency of in vitro (Felgner et al., 1987) and in vivo gene transfer (Zhu et al., 1993; Philip et al., 1993; Solodin et al., 1995; Liu et al., 1995; Thierry et al., 1995; Tsukamoto et al., 1995; Aksentijevich et al., 1996).

An exemplary method for targeting viral particles to cells that lack a single cell-specific marker has been described (U.S. Pat. No. 5,849,718). In this method, for example, antibody A may have specificity for tumor, but also for normal heart and lung tissue, while antibody B has specificity for tumor but also normal liver cells. The use of antibody A or antibody B alone to deliver an anti-proliferative nucleic acid to the tumor would possibly result in unwanted damage to heart and lung or liver cells. However, antibody A and antibody B can be used together for improved cell targeting. Thus, antibody A is coupled to a gene encoding an anti-proliferative nucleic acid and is delivered, via a receptor mediated uptake system, to tumor as well as heart and lung tissue. However, the gene is not transcribed in these cells as they lack a necessary transcription factor. Antibody B is coupled to a universally active gene encoding the transcription factor necessary for the transcription of the anti-proliferative nucleic acid and is delivered to tumor and liver cells. Therefore, in heart and lung cells only the inactive anti-proliferative nucleic acid is delivered, where it is not transcribed, leading to no adverse effects. In liver cells, the gene encoding the transcription factor is delivered and transcribed, but has no effect because no an anti-proliferative nucleic acid gene is present. In tumor cells, however, both genes are delivered and the transcription factor can activate transcription of the anti-proliferative nucleic acid, leading to tumor-specific toxic effects.

The addition of targeting ligands for gene delivery for the treatment of hyperproliferative diseases permits the delivery of genes whose gene products are more toxic than do non-targeted systems. Examples of the more toxic genes that can be delivered includes pro-apoptotic genes such as Bax and Bak plus genes derived from viruses and other pathogens such as the adenoviral E4 or f4 and the E. coli purine nucleoside phosphorylase, a so-called “suicide gene” which converts the prodrug 6-methylpurine deoxyriboside to toxic purine 6-methylpurine. Other examples of suicide genes used with prodrug therapy are the E. coli cytosine deaminase gene and the HSV thymidine kinase gene.

It is also possible to utilize untargeted or targeted lipid complexes to generate recombinant or modified viruses in vivo. For example, two or more plasmids could be used to introduce retroviral sequences plus a therapeutic gene into a hyperproliferative cell. Retroviral proteins provided in trans from one of the plasmids would permit packaging of the second, therapeutic gene-carrying plasmid. Transduced cells, therefore, would become a site for production of non-replicative retroviruses carrying the therapeutic gene. These retroviruses would then be capable of infecting nearby cells. The promoter for the therapeutic gene may or may not be inducible or tissue specific.

Similarly, the transferred nucleic acid may represent the DNA for a replication competent or conditionally replicating viral genome, such as an adenoviral genome that lacks all or part of the adenoviral E1a or E2b region or that has one or more tissue-specific or inducible promoters driving transcription from the E1a and/or E1b regions. This replicating or conditional replicating nucleic acid may or may not contain an additional therapeutic gene such as a tumor suppressor gene or anti-oncogene.

d. Lipid Administration

The actual dosage amount of a lipid composition (e.g., a liposome-adenovirus) administered to a patient can be determined by physical and physiological factors such as body weight, severity of condition, idiopathy of the patient and on the route of administration. With these considerations in mind, the dosage of a lipid composition for a particular subject and/or course of treatment can readily be determined.

The present invention can be administered intravenously, intradermally, intraarterially, intraperitoneally, intralesionally, intracranially, intraarticularly, intraprostaticaly, intrapleurally, intratracheally, intranasally, intravitreally, intravaginally, rectally, topically, intratumorally, intramuscularly, intraperitoneally, subcutaneously, intravesicularlly, mucosally, intrapericardially, orally, topically, locally and/or using aerosol, injection, infusion, continuous infusion, localized perfusion bathing target cells directly or via a catheter and/or lavage.

IV. Adenoviral Vectors

The present invention is directed to delivery of a therapeutic polynucleotide, preferably on a circular adenoviral DNA, within a liposome. In some embodiments, the adenoviral DNA is an adenovirus expression vector. A particular method for delivery of the expression constructs involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue-specific transforming construct that has been cloned therein.

In some embodiments, the expression vector comprises a genetically engineered form of adenovirus. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

A particular method of introducing a therapeutic polynucleotide to an animal is to introduce a replication-deficient adenovirus containing the therapeutic polynucleotide. The replication-deficient construct made by E1B and E3 deletion also avoids the viral reproduction inside the cell and transfer to other cells and infection of other people, which means the viral infection activity is shut down after it infects the target cell. The therapeutic polynucleotide is still expressed inside the cells. Also, unlike retrovirus, which can only infect proliferating cells, adenovirus is able to transfer the therapeutic polynucleotide into both proliferating and non-proliferating cells. Further, the extrachromosomal location of adenovirus in the infected cells decreases the chance of cellular oncogene activation within the treated animal. A skilled artisan recognizes that a “gutless” adenoviral vector may be utilized, such as a recombinant adenoviral vector that is deleted of all Ad genes. Gutless rAVs can be propagated using a helper virus. In the most efficient system to date, an E1-deleted helper virus is used with a packaging signal that is flanked by bacteriophage P1 loxP sites (“floxed”). Infection of the helper cells that express Cre recombinase with the gutless virus together with the helper virus with a floxed packaging signal should only yield gutless rAV, as the packaging signal is deleted from the DNA of the helper virus. In another specific embodiment, a gutless vector is incapable of expressing any adenovirus antigens. An example of constructing a gutless adenoviral vector is described in U.S. Pat. No. 6,228,646. Other examples are described in Hardy et al. (1996). U.S. Pat. No. 6,156,497 is directed to the rapid generation of adenoviral vectors from which all adenovirus backbone genes have been deleted. Such gutless vectors provide a significant advance over presently available vectors because the toxicity and immunogenicity of adenoviral backbone gene products is avoided. Furthermore, a skilled artisan recognizes such a vector could incorporate up to 37,200 base pairs of heterologous sequence, as opposed to the 7,000 base pair limit incurred by standard vectors.

It is advantageous if the adenovirus vector is replication defective, or at least conditionally defective. The adenovirus may be of any of the 42 different currently known serotypes or subgroups A-F. Adenovirus type 5 of subgroup C is presently preferred starting material for obtaining conditional replication-defective adenovirus vector for use in the present invention. This is because Adenovirus type 5 is a human adenovirus about which the most biochemical and genetic information is known, and it has historically been used for most constructions employing adenovirus as a vector. In a specific example, in Matsubara et al. (2001) a recombinant Ad-OC-E1a was constructed using a noncollagenous bone matrix protein osteocalcin (OC) promoter to drive the viral early E1a gene with restricted replication in cells that express OC transcriptional activity. A skilled artisan is aware that this is merely exemplary, and that a particular promoter could be selected depending on the disease and target tissue which is being treated.

Generation and propagation of the current adenovirus vectors, which are replication deficient, depend on a unique helper cell line, designated 293, which was transformed from human embryonic kidney cells by Ad5 DNA fragments and constitutively expresses E1 proteins (E1A and E1B; Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1and E3 regions, the maximum capacity of the current adenovirus vector is under 7.5 kb, or about 15% of the total length of the vector. More than 80% of the adenovirus viral genome remains in the vector backbone.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

For some embodiments, other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10⁹ to 10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991; Stratford-Perricaudet et al., 1991; Rich et al., 1993). Studies in administering recombinant adenovirus to different tissues include trachea instillation (Rosenfeld et al., 1991; Rosenfeld et al., 1992), muscle injection (Ragot et al, 1993), peripheral intravenous injections (Herz and Gerard, 1993) and stereotactic inoculation into the brain (Le Gal La Salle et al., 1993). Recombinant adenovirus and adeno-associated virus (see below) can both infect and transduce non-dividing human primary cells.

The present invention, in particular embodiments, is directed to having a circular adenovirus vector. Covalently closed circles of adenovirus 5 DNA are known in the art (Ruben et al., 1983).

In a specific embodiment, the dl1520 adenovirus is utilized in methods and/or compositions of the present invention. It has been described by McCormick (PCT/US94/02049, filed Feb. 16, 1994) that a recombinant adenovirus, d11520, produced by Barker and Berk Virology vol.156: page 107-121 (1987), selectively replicates and lyses p53 minus cancer cells but not normal cells. Moreover, newly replicated virus was shown to be competent to infect and lyse neighboring cancer cells. Thus, in at least one respect, this finding is a marked advance over current gene therapy approaches which, to be maximally effective require that all cancer cells be infected following viral infection.

V. Therapeutic Polynucleotides and Transfer Therapy Methods

Adenoviral DNA vectors in combination with an efficient delivery system of the present invention is a particularly attractive approach for gene transfer therapy. In preferred embodiments of the present invention, the adenovirus, comprised within a liposome, itself comprises a therapeutic polynucleotide for transfer into a recipient organism. Examples of therapeutic polynucleotides are well known in the art, and a skilled artisan recognizes that the particular therapeutic polynucleotide must be selected for by a health care provider for a specific therapy.

The liposomal composition of the present invention may be systematically administered into patients parenterally in order to achieve transfer therapy of one or more biologically active agents, such as an adenoviral DNA. Moreover, this technique may be used for “ex vivo” transfer therapy where tissue or cells are removed from patients, then treated and finally reimplanted in the patient. Alternatively, systemic therapy is also effective in administering the adenovirus-liposome.

Many diseases can be treated via the drug delivery system of the present invention. Diseases such as cancer, diabetes, atherosclerosis, chemotherapy-induced multi-drug resistance, and generally, immunological, neurological and viral diseases can be treated using the present drug delivery system.

In a specific embodiment, the adenoviral DNA comprising the therapeutic polynucleotide is circular. In specific embodiments, the adenovirus-liposomes comprising the nucleic acid drug can be administered by intravenous, intramuscular, intraperitoneal, subcutaneous intra-lesional and/or oral means.

A degree of tissue specific expression can be obtained depending upon the liposome preparation, the route of administration and the promoter driving expression of the transgene. Furthermore, replication of the adenovirus can be conditional, such as tissue-specific. Useful promoters are well-known to the skilled artisan and can be substantiated for those exemplified herein. It is clear that the more cationic (DNA/total lipid ratio <0.05, w/w) the liposomes, the more lung and heart are targeted. Although reporter gene expression may be lower following subcutaneous (“s.c.”) administration of liposomal DNA compared to i.v. administration, there were no changes in tissue targeting. In contrast, after intraperitoneal (“i.p.”) administration the spleen can be particularly targeted. It is also known that the CMV promoter is capable of more efficient expression in spleen than in lung when compared to the RSV promoter. No significant difference has been observed in liver and heart. Thus, the choice of the promoter may greatly influence the efficacy of non-retroviral mediated gene delivery and may lead to a certain degree of tissue specificity.

In one embodiment of the present invention, the transgene expression following a single injection of liposomal DNA was investigated, although it is also shown that repeated injection increases and/or prolongs transgene expression. Desirable transgenes are repetitively administrated and thus offer attractive alternatives to adenoviral-mediated gene therapy in the absence of the liposomes. In specific embodiments, using the adenoviral-liposomes of the present invention, administration via systemic delivery or other means, such as aerosol delivery, induces immunogenecity when the liposomal DNA composition is administrated at doses which produced detectable transgene expression.

A proposed daily dosage of active compound for the treatment of man is empirically determined by standard means in the art, but exemplary dosages are from 0.5 mg DNA/kg to 4 mg DNA/kg, which may be conveniently administered in one or two doses. The precise dose employed will of course depend on the age and conditions of the patient and on the route of administration. Thus a suitable dose for administration by inhalation is 0.5 mg DNA/kg to 2 mg DNA/kg, for oral administration is 2 mg DNA/kg to 5 mg DNA/kg, for parenteral administration is 2 mg DNA/kg to 4 mg DNA/kg.

The compound of the invention may be formulated for parenteral administration by bolus injection or continuous infusion. Formulation for injection may be presented in unit dosage form in ampules, or in multi-dose containers with an added preservative. The compositions may take such forms as suspension, solutions or emulsions in oily or aqueous vehicles, and may contain formulatory agents such as suspending, stabilizing and/or dispersing agents. Alternatively, the active ingredient may be in powder form for reconstitution with a suitable vehicle, e.g. sterile pyrogen-free water, before use.

The compounds according to the invention may be formulated for administration in any convenient way. The invention therefore includes within its scope pharmaceutical compositions comprising at least one liposomal compound formulated for use in human or veterinary medicine. Such compositions may be presented for use with physiologically acceptable carriers or excipients, optionally with supplementary medicinal agents. Conventional carriers can also be used with the present invention.

For oral administration, the pharmaceutical composition may take the form of, for example, tablets, capsules, powders, solutions, syrups or suspensions prepared by conventional means with acceptable excipients.

Certain methods of preparing dosage forms of the invention lipsomal adenoviral DNA compositions are known. See, e.g., Remington's Pharmaceutical Sciences, Mack Publishing Company, Easton, Pa., 17th edition, 1985. The composition or formulation administered will contain a predetermined quantity of the adenoviral DNA to achieve the desired therapeutic effect.

The various compositions of the present invention will preferably be used in combination with pharmaceutically acceptable excipient materials. Preferred pharmacologically acceptable excipients include neutral saline solutions buffered with phosphate, lactate, Tris, and other appropriate buffers known in the art.

In specific embodiments, viral DNA liposomal complexes are formulated for therapeutic and diagnostic administration to a patient having a neoplastic disease. For therapeutic or prophylactic uses, a sterile composition containing a pharmacologically effective dosage of one or more species of antineoplastic replication deficient adenovirus mutant DNA is administered to a human patient or veterinary non-human patient for treatment of a neoplastic condition. Generally, about 0.5-50 ug of viral DNA with liposome will be administered per treatment in an aqueous suspension. A pharmaceutically acceptable carrier or excipient is often employed in such sterile compositions. A variety of aqueous solutions can be used, e.g., water, buffered water, 0.4% saline, 0.3% glycine and the like. These solutions are sterile and generally free of particulate matter other than the desired viral DNA liposomal complex. The compositions may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride, sodium lactate, etc. Excipients which enhance transfection of cells by the complexes may be included.

Various human neoplasms comprising cells that lack p53 and/or RB functions may be treated with the appropriate liposomal viral DNA complexes. For example, a human patient or nonhuman mammal having a bronchogenic carcinoma, nasopharyngeal carcinoma, laryngeal carcinoma, small cell and non-small cell lung carcinoma, lung adenocarcinoma, hepatocarcinoma, pancreatic carcinoma, bladder carcinoma, colon carcinoma, breast carcinoma, cervical carcinoma, ovarian carcinoma, or lymphocytic leukemia may be treated by administering an effective antineoplastic dosage of an appropriate adenovirus E1b-p53⁽⁻⁾ liposomal complex. Viral DNA liposomal suspensions may be applied to neoplastic tissue by various routes, including intravenous, intraperitoneal, intramuscular, subdermal, and topical. A viral DNA liposomal suspension may be inhaled as a mist (e.g., for pulmonary delivery to treat bronchogenic carcinoma, small-cell lung carcinoma, non-small cell lung carcinoma, lung adenocarcinoma, or laryngeal cancer) or swabbed directly on a tumor site (e.g., bronchogenic carcinoma, nasopharyngeal carcinoma, laryngeal carcinoma, cervical carcinoma) or may be administered by infusion (e.g., into the peritoneal cavity for treating ovarian cancer, into the portal vein for treating hepatocarcinoma or liver metastases from other non-hepatic primary tumors) or other suitable route, including direct injection into a tumor mass (e.g., a breast tumor), enema (e.g., colon cancer), or catheter (e.g., bladder cancer). The advantages of the latter method have already been discussed.

Thus, in a specific embodiment, the therapeutic polynucleotide is for the treatment of cancer. In another specific embodiment, the therapeutic polynucleotide is for the treatment of lung cancer.

Some examples of therapeutic polynucleotides that may be used for cancer or other diseases include: p53, BRCA1, BRCA2, a BMP, such as BMP2, an IL, such as IL-2, thymidine kinase, and cytosine deaminase.

VI. Combined Therapy

A skilled artisan recognizes that the present invention provided herein in some embodiments is used in conjunction with another therapy for the individual. For example, in the embodiments wherein cancer is being treated with methods and/or compositions of the present invention, the patient may also receive standard chemotherapy, radiotherapy, surgery, and/or gene therapy treatments.

Tumor cell resistance to anti-cancer agents represents a major problem in clinical oncology. The present invention may also be used in combination with conventional therapies to improve the efficacy of chemotherapy, radiotherapy, and/or surgery. For example, the herpes simplex-thymidine kinase (HS-tK) gene, when delivered to brain tumors by a retroviral vector system, successfully induced susceptibility to the antiviral agent ganciclovir (Culver, et al., 1992). In the context of the present invention, it is contemplated that the compositions of the present invention could be used similarly in conjunction with chemotherapeutic, radiotherapeutic, or surgical intervention.

To kill cells, such as malignant or metastatic cells, using the methods and compositions of the present invention, one would generally contact a “target” cell with a adenoviral DNA/therapeutic polynucleotide/liposome composition and at least one anti-cancer agent. These compositions would be provided in a combined amount effective to kill or inhibit proliferation of the cell. This process may involve contacting the cells with the adenoviral DNA/therapeutic polynucleotide/liposome composition and the anti-cancer agent(s) or factor(s) at the same time. This may be achieved by contacting the cell with a single composition or pharmacological formulation that includes both agents, or by contacting the cell with two distinct compositions or formulations, at the same time, wherein one composition includes the adenoviral DNA/therapeutic polynucleotide/liposome composition and the other includes the anti-cancer agent.

Alternatively, the adenoviral DNA/therapeutic polynucleotide/liposome treatment may precede or follow the anti-cancer agent treatment by intervals ranging from min to weeks. In embodiments where the anti-cancer agent and adenoviral DNA/therapeutic polynucleotide/liposome are applied separately to the cell, one would generally ensure that a significant period of time did not expire between the time of each delivery, such that the anti-cancer agent and adenoviral DNA/therapeutic polynucleotide/liposome composition would still be able to exert an advantageously combined effect on the cell. In such instances, it is contemplated that one would contact the cell with both agents within about 6 h to one wk of each other and, more preferably, within about 24-72 h of each other, with a delay time of only about 48 h being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the adenoviral DNA/therapeutic polynucleotide/liposome or the anti-cancer agent will be desired. Various combinations may be employed, where adenoviral DNA/therapeutic polynucleotide/liposome is “A” and the anti-cancer agent is “B”:

| | | | | | |
|---|---|---|---|---|---|
| A/B/A | B/A/B | B/B/A | A/A/B | B/B/B/A | B/B/A/B |
| A/A/B/B | A/B/A/B | A/B/B/A | B/B/A/A | B/A/B/A | B/A/A/B |
| A/A/A/B | B/A/A/A | A/B/A/A | A/A/B/A | A/B/B/B | B/A/B/B |

To achieve cell killing, both agents are delivered to a cell in a combined amount effective to kill the cell.

In one exemplary embodiment of the present invention, the anti-cancer agent is taxol (paclitaxel). The regimen of paclitaxel administration has varied in clinical trials, the most common including a dosage of between 135 and 250 mg/m2 administered over an infusion period of 3 or 24 h once every 3 weeks (Wiseman and Spencer, 1998). Promising results have been achieved in phase I/II trials of a weekly regimen of paclitaxel (60 to 175 mg/m2). The objective response rate in patients with metastatic breast cancer (either pretreated or chemotherapy-naive) is generally between 20 and 35% with paclitaxel monotherapy, which compares well with that of other current treatment options including the anthracycline doxorubicin. Combination therapy with paclitaxel plus doxorubicin appears superior to treatment with either agent alone in terms of objective response rate and median duration of response (Wiseman and Spencer, 1998). In exemplary embodiments, the present invention contemplates the use of adenoviral DNA/therapeutic polynucleotide/liposomes combined with taxol and the use of adenoviral DNA/therapeutic polynucleotide/liposomes combined with taxol plus other anti-cancer agents such as doxorubicin.

Many anti-cancer agents are DNA damaging agents. DNA damaging agents or factors are defined herein as any chemical compound or treatment method that induces DNA damage when applied to a cell. Such agents and factors include radiation and waves that induce DNA damage such as, γ-irradiation, X-rays, UV-irradiation, microwaves, electronic emissions, and the like. A variety chemotherapeutic agents function to induce DNA damage, all of which are intended to be of use in the combined treatment methods disclosed herein. Chemotherapeutic agents contemplated to be of use, include, e.g., adriamycin, 5-fluorouracil (5FU), etoposide (VP-16), camptothecin, actinomycin-D, mitomycin C, cisplatin (CDDP) and even hydrogen peroxide. The invention also encompasses the use of a combination of one or more DNA damaging agents, whether radiation-based or actual compounds, such as the use of X-rays with cisplatin or the use of cisplatin with etoposide. Many DNA damaging agents induce apoptosis. One aspect of the present invention is the use of adenoviral DNA/therapeutic polynucleotide/liposomes to sensitize tumor cells to apoptotic agents.

In treating cancer according to the invention, one would contact the tumor cells with a DNA damaging agent in addition to the adenoviral DNA/therapeutic polynucleotide/liposome composition. This may be achieved by irradiating the localized tumor site with DNA damaging radiation such as X-rays, UV-light, γ-rays or even microwaves. Alternatively, the tumor cells may be contacted with the DNA damaging agent by administering to the subject a therapeutically effective amount of a pharmaceutical composition comprising a DNA damaging compound such as, adriamycin, 5-fluorouracil, etoposide, camptothecin, actinomycin-D, mitomycin C, or more preferably, cisplatin. The DNA damaging agent may be prepared and used as a combined therapeutic composition, or kit, by combining it with a adenoviral DNA/therapeutic polynucleotide/liposome composition, as described above.

Agents that directly cross-link polynucleotides, specifically DNA, are envisaged and are shown herein, to eventuate DNA damage leading to a synergistic antineoplastic combination. Agents such as cisplatin, and other DNA alkylating may be used. Cisplatin has been widely used to treat cancer, with efficacious doses used in clinical applications of 20 mg/m2 for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m2 at 21 day intervals for adriamycin, to 35-50 mg/m2 for etoposide intravenously or double the intravenous dose orally.

Agents that disrupt the synthesis and fidelity of polynucleotide precursors and subunits also lead to DNA damage. As such a number of polynucleotide precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of DNA damage, or the precursors of DNA, the replication and repair of DNA, and the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The inventor proposes that the regional delivery of adenoviral DNA/therapeutic polynucleotide/liposome compositions to patients with tumors will be a very efficient method for delivering a therapeutically effective gene to counteract the clinical disease. Similarly, the chemotherapy, radiotherapy, or surgery may be directed to a particular, affected region of the subject's body. Alternatively, systemic delivery of the adenoviral DNA/therapeutic polynucleotide/liposome or the DNA damaging agent may be appropriate in certain circumstances, for example, where extensive metastasis has occurred.

Cytokine therapy also has proven to be an effective partner for combined therapeutic regimens. Various cytokines may be employed in such combined approaches. Examples of cytokines include IL-1a IL-1β, IL-2, IL-3, IL-4, IL-5, IL-6, IL-7, IL-8, IL-9, IL-10, IL-11, IL-12, IL-13, TGF-β, GM-CSF, M-CSF, G-CSF, TNFa, TNFβ, LAF, TCGF, BCGF, TRF, BAF, BDG, MP, LIF, OSM, TMF, PDGF, IFN-a, IFN-β, IFN-γ. Cytokines are administered according to standard regimens, as described below, consistent with clinical indications such as the condition of the patient and relative toxicity of the cytokine.

A number of polypeptides are known to induce apoptosis and may be used in the combination therapies of the present invention or as the therapeutic polynucleotide itself. In one embodiment, the combination therapy is the use of adenoviral DNA/therapeutic polynucleotide/liposome with a polypeptide form the tumor necrosis factor (“TNF”) family. In a preferred embodiment, the TNF polypeptide is TNFα. Other polypeptide inducers of apoptosis that may be used in the present invention include, but are not limited to, p53, Bax, Bak, Bcl-x, Bad, Bim, Bik, Bid, Harakiri, Ad E1B, Bad and ICE-CED3 proteases.

VII. Pharmaceutical Compositions and Routes of Administration

Adenoviral DNA/therapeutic polynucleotide/liposome compositions of the present invention will have an effective amount of a gene for therapeutic administration, and, in some embodiments, is used in combination with an effective amount of a compound (second agent) that is an anti-cancer agent, as exemplified above. Such compositions will generally be dissolved or dispersed in a pharmaceutically acceptable carrier or aqueous medium. The term “effective” as used herein refers to providing inhibition of proliferation of at least one cell, such as in a human; providing retardation of growth of a tumor; providing shrinking in size or eradication of a tumor; providing impeding metastases; and/or providing amelioration of a cancer symptom, and so forth.

The phrases “pharmaceutically or pharmacologically acceptable” refer to molecular entities and compositions that do not produce an adverse, allergic or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredients, its use in the therapeutic compositions is contemplated. Supplementary active ingredients, such as other anti-cancer agents, can also be incorporated into the compositions.

In addition to the compounds formulated for parenteral administration, such as intravenous or intramuscular injection, other pharmaceutically acceptable forms include, e.g., tablets or other solids for oral administration; time release capsules; and any other form currently used, including cremes, lotions, mouthwashes, inhalants and the like.

The expression vectors and delivery vehicles of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra.

The compositions of the present invention are advantageously administered in the form of injectable compositions either as liquid solutions or suspensions; solid forms suitable for solution in, or suspension in, liquid prior to injection also may be prepared. These preparations also may be emulsified. A typical composition for such purposes comprises a 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters, such as theyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components in the pharmaceutical are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

An effective amount of the therapeutic agent is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual.

All of the essential materials and reagents required for inhibiting tumor cell proliferation may be assembled together in a kit and housed in a suitable container. When the components of the kit are provided in one or more liquid solutions, the liquid solution preferably is an aqueous solution, with a sterile aqueous solution being particularly preferred.

For in vivo use, a chemotherapeutic agent may be formulated into a single or separate pharmaceutically acceptable syringeable composition. In this case, the container means may itself be an inhalant, syringe, pipette, eye dropper, or other such like apparatus, from which the formulation may be applied to an infected area of the body, such as the lungs, injected into an animal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container means. The kits of the invention may also include an instruction sheet defining administration of the gene therapy and/or the chemotherapeutic drug.

The kits of the present invention also will typically include a means for containing the vials in close confinement for commercial sale such as, e.g., injection or blow-molded plastic containers into which the desired vials are retained. Irrespective of the number or type of containers, the kits of the invention also may comprise, or be packaged with, an instrument for assisting with the injection/administration or placement of the ultimate complex composition within the body of an animal. Such an instrument may be an inhalant, syringe, pipette, forceps, measured spoon, eye dropper or any such medically approved delivery vehicle.

The active compounds of the present invention will often be formulated for parenteral administration, e.g., formulated for injection via the intravenous, intramuscular, subcutaneous, or even intraperitoneal routes. The preparation of an aqueous composition that contains a second agent(s) as active ingredients will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The active compounds may be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In certain cases, the therapeutic formulations of the invention could also be prepared in forms suitable for topical administration, such as in cremes and lotions. These forms may be used for treating skin-associated diseases, such as various sarcomas.

Upon formulation, solutions will be administered in a manner compatible with the dosage formulation and in such amount as is therapeutically effective. The formulations are easily administered in a variety of dosage forms, such as the type of injectable solutions described above, with even drug release capsules and the like being employable.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

Targeting of cancerous tissues may be accomplished in any one of a variety of ways. Plasmid vectors and retroviral vectors, adenovirus vectors, and other viral vectors all present means by which to target human cancers. The inventors anticipate particular success for the use of liposomes to target therapeutic polynucleotides to cancer cells. For example, DNA encoding p53 may be complexed with liposomes in the manner described above, and this DNA/liposome complex injected into patients with certain forms of cancer, such as lung cancer; intravenous injection can be used to direct the gene to all cell. Directly injecting the liposome complex into the proximity of a cancer can also provide for targeting of the complex with some forms of cancer. For example, cancers of the ovary can be targeted by injecting the liposome mixture directly into the peritoneal cavity of patients with ovarian cancer. Of course, the potential for liposomes that are selectively taken up by a population of cancerous cells exists, and such liposomes will also be useful for targeting the gene.

Those of skill in the art will recognize that the best treatment regimens for using adenoviral DNA/therapeutic polynucleotide/liposomes to suppress tumors can be straightforwardly determined. This is not a question of experimentation, but rather one of optimization, which is routinely conducted in the medical arts. The in vivo studies in nude mice provide a starting point from which to begin to optimize the dosage and delivery regimes. The frequency of injection will initially be once a wk, as was done some mice studies. However, this frequency might be optimally adjusted from one day to every two weeks to monthly, depending upon the results obtained from the initial clinical trials and the needs of a particular patient. Human dosage amounts can initially be determined by extrapolating from the amount of adenoviral DNA/therapeutic polynucleotide/liposomes used in mice. In certain embodiments it is envisioned that the dosage may vary from between about 1 mg therapeutic polynucleotide DNA/Kg body weight to about 5000 mg therapeutic polynucleotide DNA/Kg body weight; or from about 5 mg/Kg body weight to about 4000 mg/Kg body weight or from about 10 mg/Kg body weight to about 3000 mg/Kg body weight; or from about 50mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher does may be used, such doses may be in the range of about 5 mg therapeutic polynucleotide DNA/Kg body to about 20 mg therapeutic polynucleotide DNA/ Kg body. In other embodiments the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

The present Example comprises reagents and procedures that are well known to one of skill in the art, as previously known to an artisan and as described herein. In specific embodiments, Example 1 describes methods and materials for experiments in Examples 2-9.

Cells

HEK293 (human embryonic kidney), A459 (human lung cancer), CHO (Chinese hamster ovary), and NIH-3T3 (Swiss mouse embryonic cells) cell lines were purchased from ATCC (American Type Culture Collection, Rockville, Md.). HEK293, CHO, A549 and NIH3T3 cells were maintained in Dulbecco modified Eagle's medium (DMEM, BioWhittaker, Walkersville, Md.). Media were supplemented with 10% fetal calf serum (FCS, Hyclone, Logan, Utah), 100 unit/ml penicillin, 100 μg/ml streptomycin (Life Technologies Inc., Gaithersburg, Md.), and cells were maintained at 37° C., 5% CO₂ in a humidified incubator.

Vectors

E1, E3 deleted adenovirus type 5 expressing Lac-Z, eGFP, or human α1-anti trypsin (hAAT) proteins were used in these studies. Each vector was produced by calcium phosphate transfection of HEK293 cells, followed by expansion of a single plaque. At maximal cytopathic effect, the cells were harvested and pelleted. Vectors were extracted from the HEK293 cells by 3 consecutive freeze/thaw cycles and amplified by infection of a larger culture of HEK293 cells. The vectors were purified by 2 cesium chloride gradient ultracentrifugation steps and desalted on exclusion columns (Bio-Rad Laboratories, Hercules, Calif.). The titer of the large-scale preparation was established by plaque assay using HEK293 cells. Preparations were routinely tested for replication-competent adenovector (RCA) by plaquing on A549 cells. Titer was always less than 1 RCA/10¹⁰ vp.

Preparation of Adenovirus/Liposome Complexes and Infection of Target Cells

Target cells were seeded in a 6 well plate to reach 70% confluency the next day. Liposome/virus complexes were prepared fresh at room temperature. Liposomes (DOTAP and DOTAP:chol) were formulated, including manual extrusion through Whatman ANOTOP filters as described previously (Templeton et al., 1997). DOTAP (20 mM) stock solution was diluted to a 4 mM final concentration in a 300 μl final volume with 5% dextrose in water (D5W) and adenovirus stock solution. To determine the equivalence of liposome complex and uncoated viral particle (vp) in transduction, sensitive target cells (293) were exposed to AdGFP or to AdGFP complexed to DOTAP. Maximum infectivity was found with 10³ Ad-GFP vp/cell and AdGFP/DOTAP made from 2.3×10¹⁰ vp/300 μl of 4 mM DOTAP to give a final concentration of 10³ vp/cell. All infections were performed in serum free DMEM at 37° C. Six hours post infection, the cells were washed with 4 ml of PBS and fresh medium supplemented with 10% FCS.

Flow Analysis

Twenty four hours after infection with Ad-GFP, cells were washed in PBS and analyzed by FACS. Dead cells and debris were excluded from analysis by using propidium iodide (PI). GFP expression was measured using a standard filter setup for fluorescein (525 nm, bandpass filter).

Electron Microscopy

Lipid-vector complexes were processed for cryo-electron microscopy using a negative stain/rotary shadow technique. Liposomes (at 4 mM final) were complexed to adenovirus (1/10 final dilution of a 5×10 ¹² vp/ml stock). The samples were vitrified in a quantifoil grid (R2/2, Quantifoil Micro Tools GmbH, Jena, Germany) with established procedure (Dubochet et al., 1988). The frozen, hydrated specimens were kept at −165° C. with a Gatan cryo-holder and imaged in a JEOL1200 electron cryomicroscope under low dose condition (<10 electrons/A²) at 40,000× magnification² (McGough et al., 1997). The images were digitized with a Zeiss Photodise scanner and displayed with the EMAN software (Ludtke et al., 1999).

Neutralization of Virus/Liposome by Serum

The serum of healthy human donors previously selected for a high titer of neutralizing anti adenoviral antibodies was collected and stored at −80° C. Serum was decomplemented at 56° C. for 30 min and diluted in DMEM. 293 and CHO cells were plated at 2×10⁴ cells/well in 96-well plates, cultured in DMEM for 24 hours and washed. Ad-lac-Z (for a final dilution of 10⁶ pfu/well) either alone, or complexed with liposomes as described above, was incubated for 1 hour at 37° C. with decomplemented serum pure or diluted (two-fold increments, starting at ½) in a final volume of 100 μl at 37° C. After incubation, each sample was applied to cells and incubated for 6 hours. Vector solution was then removed and replaced by 200 μl of fresh 10% FCS DMEM. Twenty-four hours after infection, the efficiency of infection was estimated for each well using a β-Galactosidase Enzyme Assay kit (Promega; Madison, Wis.). The absorbance was read at 410 nm with a spectrophotometer. Results are reported as the percentage of the maximum absorbance obtained with free virus non-inhibited by serum.

Gene Expression in Lung and Liver Tissues after Re-Injection of Ad-lac-Z/Liposome Complexes

Seven-week-old C57 B1/6J mice were obtained from the Jackson laboratory, and injected over 5 min. in the tail vein with 10⁹ pfu of Ad-lac-Z alone or Ad-lac-Z/liposome complexes in a final volume of 100 μl. Thirty days after injection the mice were re-injected under the same conditions, and followed for an additional 30 days. A pool of mice was analyzed 7 and 30 days after the first injection, and 30 days after the second injection. Mice were anesthetized with a mix of ketamine HCL (150 mg/ml) and xylazine (10 mg/ml) injected intraperitoneally (i.p.). Mice were then perfused through the right ventricle with PBS to wash out the blood. The livers and lungs were harvested intact, and embedded in OTC. Cryostat sections of 10 μm were fixed and stained with X-gal. Ten sections per organ were analyzed. Each experiment was performed three times.

Blood from each mouse was removed from the retro-orbital plexus, and after clotting and centrifugation, the serum was stored at −80° C.

X-Gal Staining

Cells: 293 cells were washed, fixed at 4° C. for 30 min with PBS containing 1.8% formaldehyde and 2% glutaraldehyde, washed in PBS, then incubated at 37° C. overnight with X-Gal substrate (20 mM NapH₂PO₄, 250 mM Na₂HPO₄, 1.3 mM MgCl₂, 3 mM K₃Fe(CN)₆, 3 mM K₄Fe(CN)₆, 1 mg/ml X-gal (dissolved in dimethylformamide) in H₂O) at 37° C. in a humidified chamber.

Tissues: X-gal staining was performed on 7 μm frozen sections. Tissues were cut, slides were fixed, washed in PBS and stained with the X-Gal solution overnight at 37° C.

Human α-1 Anti Trypsin Expression in the Plasma of Injected Mice

Groups of 5 C57 B1/6J mice were injected over 5 min. in the tail vein with a) 10⁹ pfu of Ad-hAAT; b) 10⁹ pfu of Ad-hAAT pre-incubated with neutralizing human serum (v/v); c) 10⁹ pfu of Ad-hAAT/liposome complexes; or d) 10⁹ pfu (pfu:vp=20:1) of Ad-hAAT/liposome complexes pre-incubated with human neutralizing serum (v/v). In all groups, mice were injected with a final volume of 200 μl. One week after injection the mice were re-injected under the same conditions. The blood of each mouse was harvested every 3-4 days by retro-orbital sampling and the serum was tested by enzyme-linked-immunosorbent assay (ELISA) for hAAT level as previously described by Kay et al (1992) and Morral et al (1997). Microplates were coated with 1 μg/ml of goat anti-hAAT (Cappel, Durham, N.C.) for 1 h at 37° C. Non-specific binding was blocked by overnight incubation at 4° C. with TBS-Tween-20 (0.05 M Tris pH7.5, 0.1 M NaCl, 0.05% Tween-20) mixed with non-fat dry milk. Samples were diluted and incubated at 37° C. for 2 h. After washing, the plates were incubated with horseradish peroxidase (HRP)-conjugated goat-anti-hAAT (Cappel, Durham, N.C.) for 2.5 h at 37° C. After further washing, the substrate was added to the wells and the plates incubated at RT in the dark. The reaction was stopped with 2N H₂SO₄ and optical density was read at 450 nm.

Analysis of Anti-Ad-hAAT Antibody Production

Sera from mice were assessed for specific Ad-hAAT by ELISA as described by Kay et al and Morral et al (Kay et al,. 1992; Morral et al., 1997). Briefly, microplates (MaxiSorp, Nunc) were coated with UV-inactivated Ad-hAAT (1×10⁸ vp in 50 μl of 0.1M NaHCO₃/well) at 4° C. overnight, washed and blocked for 1 h at room temperature (RT). The wells were washed, and diluted serum (3-fold dilutions with blocking buffer beginning at 1:10) was added to the wells and incubated at RT for 2 h. The plates were washed and incubated with HRP-conjugated goat anti-mouse (Sigma) for 2 h at RT. The plates were washed again, incubated with substrate solution and incubated in the dark, and the reaction stopped by the addition of 2N H₂SO₄.

Neutralizing Antibody Titers

Sera from adenovirus-injected mice were tested for their ability to inhibit adenovirus infection. Dilutions of each serum were incubated with Ad-Lac-Z virus and incubated for 1H at 37° C. Each dilution was then added to 293 cells (2-4 10⁴ cells per well) and incubated at 37° C. Twenty-four hours post-infection, the cells were analyzed for Lac-Z expression. The titer was determined by the highest dilution at which the serum inhibited more than 50% of infectivity compared to the control well without serum.

Inflammatory Cytokines

Serum was collected from injected mice 6 hr and 24 hr after injection of vectors and analyzed for the production of IL6 by commercial ELISA following the manufacturer's instructions (Cell Sciences; Inc. Norwood, Mass.; PharMingen; San Diego, Calif.).

### Example 2

Electron Microscopy of the Vector/Liposome Complex

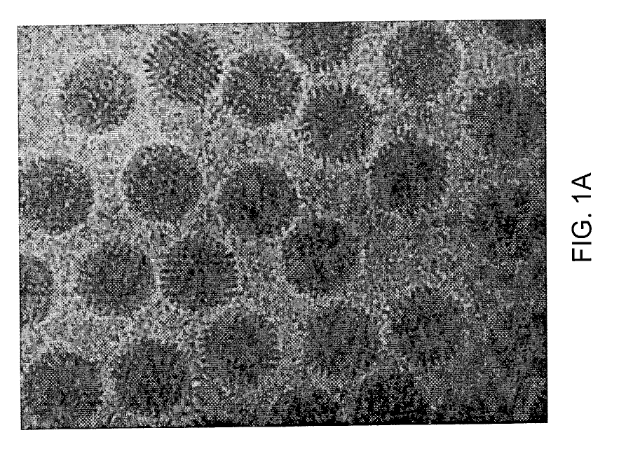

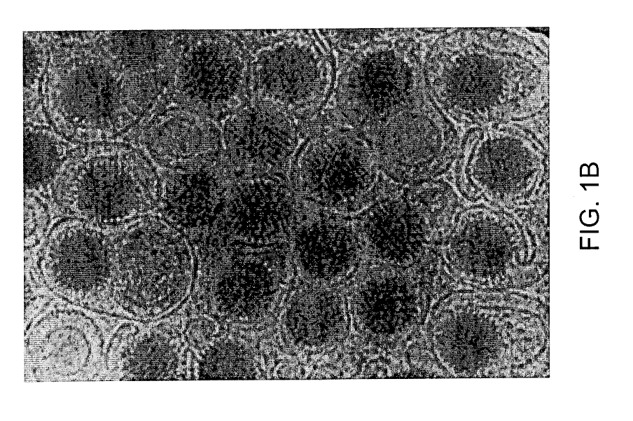

The structure of the liposome/vector complex was examined by electron microscopy using negative staining. FIG. 1 shows photomicrographs of adenovector particles complexed (FIG. 1A) or not (FIG. 1B) with liposomes. After addition of liposomes, the vector (5×10⁸ vp/μl) is completely encapsulated, and few if any free particles are detected.

### Example 3

Encapsulation Modifies Viral Target Cell Range

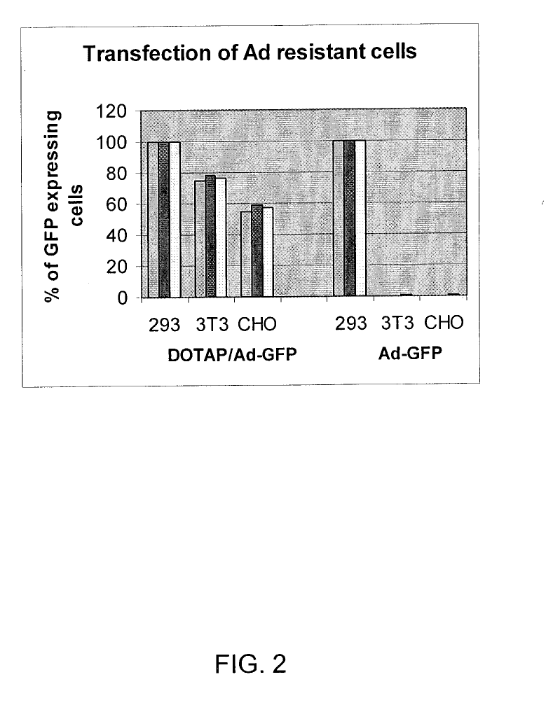

Ad5 does not infect NIH3T3 and CHO cells because they lack CAR expression (Fasbender et al., 1997; Seth et al., 1994). To determine if liposomal encapsulation allowed the adenovector to be transported into these CAR negative cells and subsequently expressed, CAR positive (293 cells) and CAR negative cells (CHO and NIH3T3) were infected with Ad-GFP alone and Ad-GFP/liposome complexes in three independent experiments. Both the vector alone (10³ Ad-GFP vp per cell) and the liposome-complexed vector (complex formed with 2.3×10¹⁰ _Ad-GFP vp/300 μl of 4 mM DOTAP, to give 10³ vp per cell) produced essentially 100% positivity in CAR expressing 293 cells (FIG. 2). Titration data obtained using a serial dilution of both Ad5-GFP and Ad5-GFP/liposome to infect 293 cells showed equivalence between uncoated vp and DOTAP-vp. Indeed, at very low vp/cell, DOTAP complexes produced superior efficiency to adenovirus infection (Table 2).

In contrast, the CAR negative cell line could be transduced only when exposed to Ad-GFP/liposomes (FIG. 2).

TABLE 2
Effect of viral particle number on percentage infection of 293 cells.

| VP/well | 10⁹ | 10⁸ | 10⁷ | 10⁶ | 10⁵ | 10⁴ |
|---|---|---|---|---|---|---|
| Ad-GFP | 99 ± 0 | 94 ± 0.5 | 90 ± 3 | 32 ± 0.5 | 7 ± 0.5 | 2 ± 0.5 |
| DOTAP-Ad-GFP | 94 ± 0.9 | 80 ± 1.5 | 59 ± 0.1 | 47 ± 1.5 | 42 ± 1.5 | 39 ± 2 |

293 cells were seeded in a 6 well plate. After 24 h of culture, 80% confluent cells were infected in serum free medium with virus alone or complexed to DOTAP. After 6 hr incubation at 37° C. for 6H, the cells were washed and placed in fresh media containing 10% serum. Results are expressed as the percentage of GFP expressing cells (mean value±SEM n=6) 24 h post-infection.

### Example 4

Inhibition of Infection in vitro in the Presence of Neutralizing Antibodies

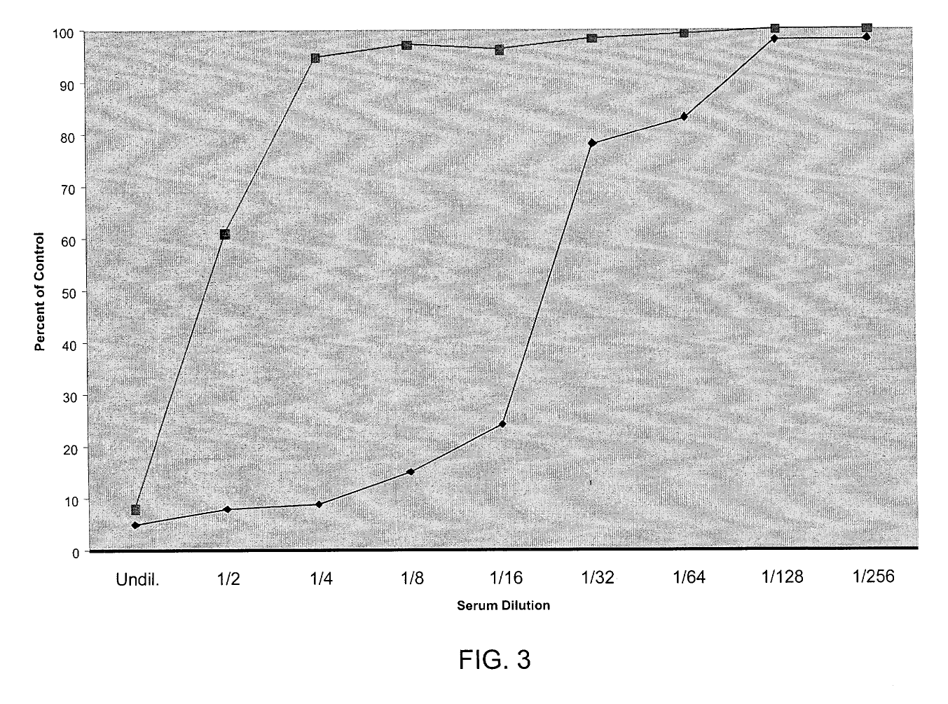

It was next assessed whether these encapsulated vectors were protected by the bilamellar coating from high titer neutralizing antibodies present in the serum of immune humans. Serum from 10 healthy donors was assessed for their capacity to inhibit adenovector infection of 293 cells and used the one with the highest titer. Both 293 and CHO cells were exposed to free vector or vector-complexed with liposomes, in the presence and absence of neutralizing serum. 293 cells infected with 10³ vp of vector produced β-Galactosidase, but infectivity after pre-incubation with neutralizing serum was dramatically reduced compare to the expression measured in the absence of neutralizing antibodies (FIG. 3). There was little infection of (CAR negative) CHO cells by vector alone, with or without pre-incubation with neutralizing serum. As in previous experiments, both 293 and CHO cell lines produced the transgene after infection with the vector complexed with liposomes (FIG. 3). Moreover, incubation with the neutralizing serum did not affect the efficiency of the vector/liposome complex infection (only at very high serum concentration). Instead, both cell lines were highly positive for β-Galactosidase production in the presence or absence of neutralizing antibody. These results confirm the EM observation that the vector particles are not exposed after bilamellar encapsulation and are protected from antibody neutralization. Vectors complexed with the conventional liposome Lipofectamine (DOSPA:DOPE) failed to display the protective effect of the DOTAP complexes (FIG. 3).

### Example 5

β-Glactosidase Expression in Re-Injected Mice

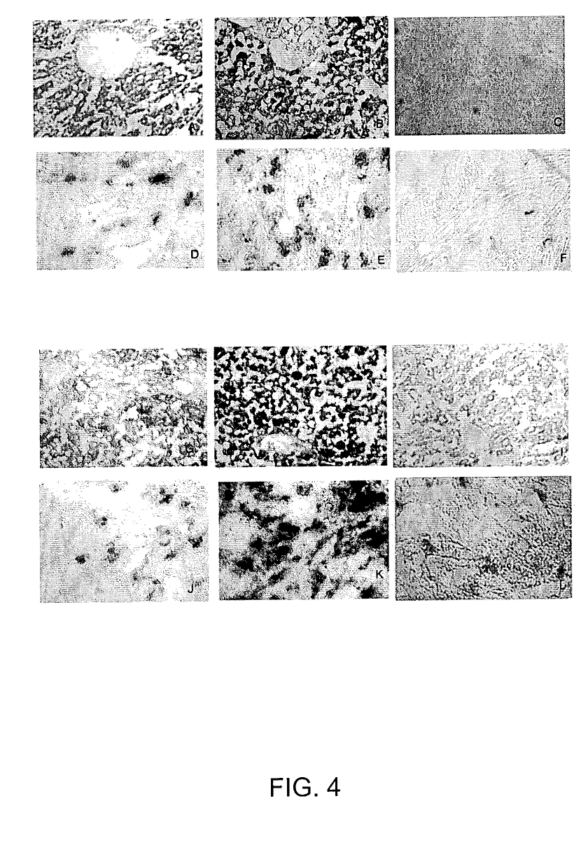

To evaluate liposome protection from antibody in vivo, 57B1/6J mice were challenged with Ad-lac-Z alone or encapsulated in liposomes. Mice were injected in the tail vein, with half of the mice in each group receiving a second identical injection one month later. Mice were sacrificed one month following either the first or the second vector injection and the livers and lungs stained for β-galactosidase expression. The liver of mice injected with virus alone showed a high level of β-galactosidase expression at day 7 and a lower expression at day 30 post injection (FIGS. 4A and 4B). By day 60, expression was undetectable, even in mice that received a second injection of uncoated vp at day 30 (FIG. 4C). In the lungs of these mice, β-galactosidase expression was detected at day 7 (FIG. 4D), but had greatly decreased by day 30 (FIG. 4E), and by day 60, no blue cells were detected in the lungs of any animals so treated (FIG. 4F).

Mice that received DOTAP:chol/Ad-lac-Z expressed β-galactosidase in their liver up to 30 days (FIGS. 4G, and 4H). This expression disappeared at day 60 but in contrast to the vector alone group, β-expression returned if the animals were re-injected with the complex on day 30 (FIG. 4I). Similarly, lungs from these mice expressed β-galactosidase both at day 7 (FIG. 4J) and day 30 (FIG. 4K). While gene expression then declined substantially, re-expression was readily apparent if the animals received a second injection with liposome encapsulated advector at day 30 (FIG. 4L).

Of note, this maintained susceptibility to re-infection by the vector-liposome complexes did not come about because the complexes were non-immunogenic. On the contrary, analysis of sera from both groups of injected animals showed that the titer of anti-adenoviral Ab was higher in the serum of the liposome/vector-injected group than in mice receiving vector alone (Table 2). Hence, the ability to transduce in vivo with liposome complexes occurs despite the presence of a high titer of murine anti-adenoviral antibodies, rather than because of their absence.

### Example 6

α1-Anti Trypsin (hAAT) Level in the Serum of Mice Immunized in Presence/Absence of Human Neutralizing Serum (NS)

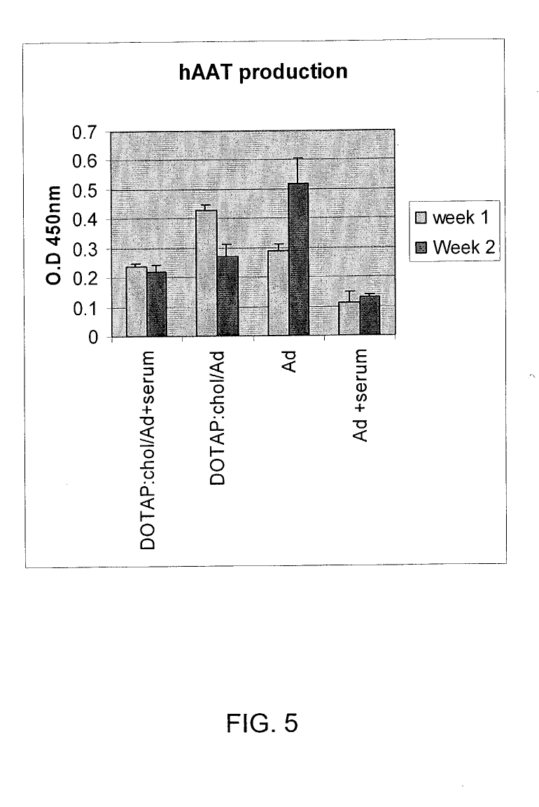

To estimate the likely effects of pre-existing human neutralizing anti-adenoviral antibodies on the function of encapsulated versus naked adenoviral vectors, mice were injected with i.v. DOTAP:chol/Ad-hAAT or Ad-hAAT with an added neutralization step. Mice were immunized with virus alone (vs⁻), virus plus neutralizing serum (vns⁺), virus/liposomes (lns⁻), virus/liposomes plus neutralizing serum (lns⁺). Control animals received an injection of PBS (ns⁻). One week after the first injection the mice received a second identical injection. For each group the blood was collected at different time points and analyzed for the presence of circulating hAAT. FIG. 5 shows that after the first injection, the level of hAAT is highest in the group receiving DOTAP:chol (lns⁻) (p<0.05). This level was decreased by adding serum (lns⁺), but stays significantly higher than the level of hAAT detected in the group injected with non-complexed-virus plus ns⁺ (vns⁺) (p<0.05). After the second injection, antibody has no significant effect on hAAT levels in the mice receiving liposomes (FIG. 5) (lns⁺), but markedly affects the uncoated vector (lns⁻) (p<0.05).

### Example 7

Ad-hAAT Antibodies in the Serum of Immunized Mice

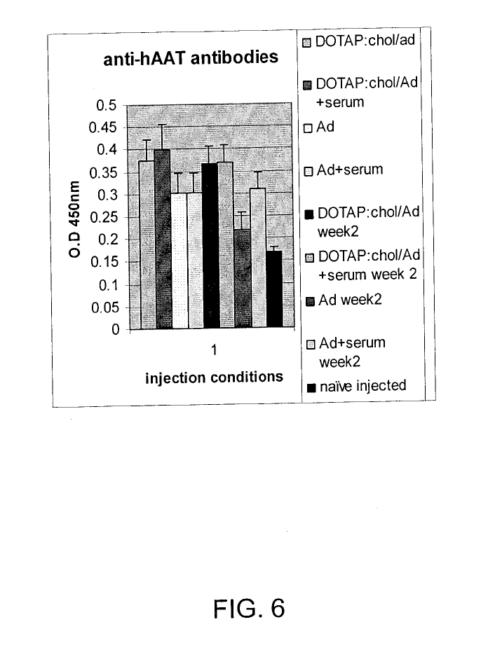

The mice injected as described above were tested by ELISA for the presence of anti-adenovirus antibodies. FIG. 6 shows that after the first and second injection there is a higher level of anti-Ad-hAAT antibodies in the animals injected with liposomes. These results are consistent with the data obtained after Ad-lac-Z/liposome injection shown previously (Table 2). This effect was observed independently of the presence of human neutralizing serum. These antibodies were able to neutralize uncoated virus as shown in Table 4 where the group receiving DOTAP:chol/Ad-hAAT generate a high level of neutralizing antibody, that (like human neutralizing antibody) is evidently unable to inhibit the effectiveness of coated adenovectors.

TABLE 3
Titration of neutralizing antibodies in the serum of Ad-lac-z injected mice.

| | Ad-GFP | DOTAP:Chol/Ad-GFP |
|---|---|---|
| D3 after 1^{st} injection | 1/4 | 1/4 |
| D7 after 1^{st} injection | 1/4 | 1/4 |
| D15 after 1^{st} injection | 1/4 | 1/16 |
| D30 after 2^{nd} injection | 1/4 | 1/512 |

Three, seven and fifteen days after the first injection and 30 days after the second injection, the serum of mice was analyzed for the presence of neutralizing antibody using the assay described in the Methods section. Results are expressed as the serum dilution blocking 293 infection by >70%.

### Example 8

Inflammatory Response to Adenovirus

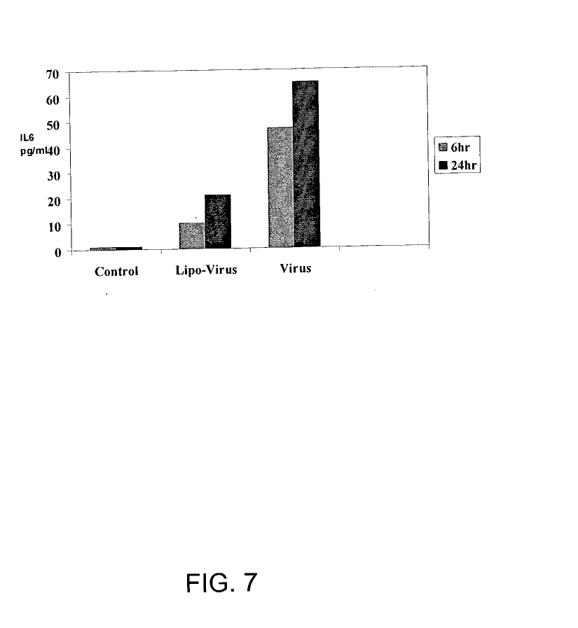

To determine whether injection of liposome coated and uncoated adenovectors in functionally equivalent amounts induced an identical inflammatory response, serum IL6 was measured in each animal 6 h after i.v. injections of each vector preparation. There was a substantial increase in serum IL6 in mice injected with virus alone that was significantly greater than mice receiving an equivalent dose of virus plus liposome (p<0.02) (FIG. 7).

TABLE 4
Neutralizing anti-Adenovector antibody in the serum of immunized mice.

| | Week 1 | Week 2 |
|---|---|---|
| DOTAP:chol/Ad-hAAT | 1/8 | 1/64 |
| DOTAP:Chol/Ad-hAAT + serum | 1/2 | 1/138 |
| Ad-hAAT | 1/8 | 1/32 |
| Ad-hAAT + serum | 1/2 | 1/8 |

The serum of mice (n=5 in each group) injected 28 days previously with Ad-hAAT or liposome/Ad-hAAT was mixed with Ad-lac-z and incubated for 30 minutes at 37° C., then added to 293 cells. Infection was stopped 6 hrs after incubation. The β-galactosidase was measured after 24 hrs. Results are expressed as the highest serum dilution allowing >70% infection of 293.

### Example 9

Circulation of Adenoviral Vectors

The adenoviral DNA is circularized by standard means in the art. A skilled artisan recognizes that routine molecular biology and cloning techniques are described in Sambrook et al. (1989) and Ausubel et al. (1994), both of which are incorporated here by reference.

### Example 10

Materials and Methods

The present Example comprises reagents and procedures that are well known to one of skill in the art, as previously known to an artisan and as described herein. In specific embodiments, Example 10 describes experiments in Examples 11-17.

Cells

HEK293 (human embryonic kidney), A459 (non-small cell lung carcinoma, wt p53), H1299 (non-small cell lung carcinoma, p53-null), cell lines were purchased from ATCC (American Type Culture Collection, Rockville, Md.) T24 (human bladder cancer) was a gift from Dr F. Marini (M. D. Anderson Cancer Center; Houston, Tex.). HEK293, T24 and A549 cells were maintained in Dulbecco modified Eagle's medium (DMEM, BioWhittaker, Walkersville, Md.), H1299 in RPMI (BioWhittaker, Walkersville, Md.). Media were supplemented with 10-15% fetal calf serum (FCS, Hyclone, Logan, Utah), 100 unit/ml penicillin, 100 μg/ml streptomycin (Life Technologies Inc., Gaithersburg, Md.) and cells were maintained at 37° C., 5% CO₂ in a humidified incubator.

Vector

Circular dl1520 was based on the pFG140 and pAd5#5-bfp plasmids, and on dl1520 viral DNA. pFG140, described by Graham (Graham, 1984; Graham et al., 1989) is an infectious Ad5 circular DNA derived from Ad5d1309. It comprises an E3 deletion and an ampicillin resistance gene as well as a bacterial origin of replication at bp1339.

The 2.3 Kb XbaI-XbaI fragment from pFG140 plasmid was inserted in the XbaI site of a Ad5#5-bfp plasmid deleted of its 2 Kb XbaI-XbaI fragment. The Sse-Sse fragment of the Ad5#5 bfp-(XbaI-XbaI 2.3 Kb PFG140) was extracted and the 3.9 Kb Sse-XbaI fragment from PFG140 inserted, and the ClaI-EcoRI fragment was replaced by the ClaI-EcoRI fragment of the dl1520 viral DNA. The plasmid was expanded in BJ5183 bacteria, and the clone used to retransform stable-2 bacteria for amplification. Working stocks of dl1520 virus were grown in 293 cells, purified on a CsCl gradient, and titered on 293 cells.

Liposome Preparation

Liposome/virus complexes were prepared fresh at room temperature. Liposomes (DOTAP and DOTAP:chol) were formulated, including manual extrusion through Whatman ANOTOP filters, as described previously (Templeton et al., 1997; U.S. Pat. No. 6,413,544. DOTAP (20 mM) stock solution was diluted to a 4 mM final concentration in a 300 μl final volume with 5% dextrose in water (D5W) and adenovirus or plasmid stock solution. To determine the equivalence of liposome complex and circular plasmid in transduction, sensitive target cells (293) were exposed to circular dl1520 complexed to DOTAP. All in vitro transfections were performed in serum-free DMEM at 37° C. Six hours post transfection, the cells were washed with 4 ml of PBS, and then fresh medium supplemented with 10% FCS was added.

In Vitro Cytopathic Effect Assays (CPE)

CPE assay was carried as described (Yotnda et al., 2002). Briefly, cells (293 and H1299) were grown in 6-well plates at a concentration of 5×10⁵ cells/well until they reached 70-90% confluency. Cultures were treated with an increasing dose of virus (up to 10³ vp/cell) or DOTAP/plasmid (5-10 μg/well) of dl1520 viral DNA, PFG140, E1⁻-Ad-GFP or circular adenovirus DNA. Plates were monitored for cytopathic effects every other day.

Virus Production in p53⁻ Cells

H1299 were seeded and transfected as described above. After 72 hours, the supernatant and the floating cells were pooled with the trypsinized cells, washed in PBS and suspended in 20 mM Tris, pH 7.4. Viruses were extracted from the cells by 3 consecutive freeze/thaw cycles and digested by XhoI or titrated (vp/ml) by measuring the optical density (OD₂₆₀).

Neutralization of Virus by Serum

The serum of healthy human donors previously selected for a high titer of neutralizing anti adenoviral antibodies was collected and stored at −80° C. Serum was decomplemented at 56° C. for 30 min and diluted in DMEM. 293 and H1299 cells were plated in 6-well plates, cultured in DMEM for 24 hours, and washed. dl1520 virus either alone or complexed with liposomes as described above and DOTAP/circular dl1520 were incubated for 1 hour at 37° C. with decomplemented serum 1/8 diluted. After incubation, each sample was applied to cells and incubated for 6 hours. Vector solution was then removed and replaced by fresh 10% FCS DMEM. The efficiency of infection was estimated for each well by observing CEP effect.

In vivo Murine Model

A xenograft model of localized disease was utilized. H1299 cells (non-small cell human lung carcinoma, p53-null) were administered subcutaneously (s.c) to SCID/beige mice (Harlan Sprague-Dawley; Indianapolis, Ind.) or to Nu/nu athymic mice (Harlan Sprague-Dawley; Indianapolis, Ind.). Mice were injected with 4-10×10⁶ H1299 cells subcutaneously in the back. Tumor bearing mice were injected intraveinously (i.v) or intratumorally (i.t) with 50-100 μg of dl1520 circular DNA/liposome, linear viral DNA/liposome, or with virus. dl1520 virus and an irrelevant-plasmid/liposome or liposome alone were also used as controls. Mice were injected each two days for 7 to 11 weeks (depending on the experiments). Tumor growth inhibition was evaluated in each group (n=7) by measuring the tumors.

In a second series of experiments, cells were previously transfected with the circular dl1520/DOTAP in vitro before injecting the mice.

Immunohistochemistry for Adenovirus Hexon/E1a Protein

Immunohistochemical analysis of formalinized tumor sections used staining with the primary antibody (anti-Hexon) at 1:1000 dilution. After 1 h of incubation, the sections were washed with a biotinylated secondary antibody followed by streptavidin-horseradish peroxidase conjugate. Diaminobenzidine was used as the chromogen and the sections were then counterstained with hematoxylin.

### Example 11

Construction of an Infectious Circular dl1520 DNA

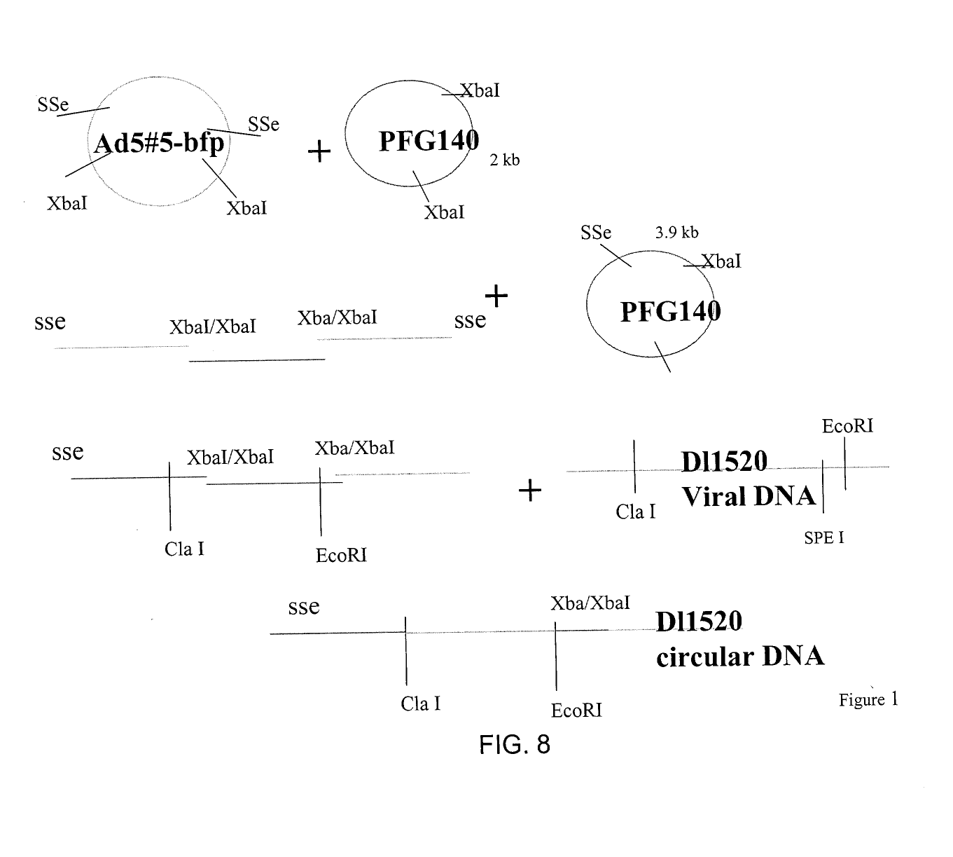

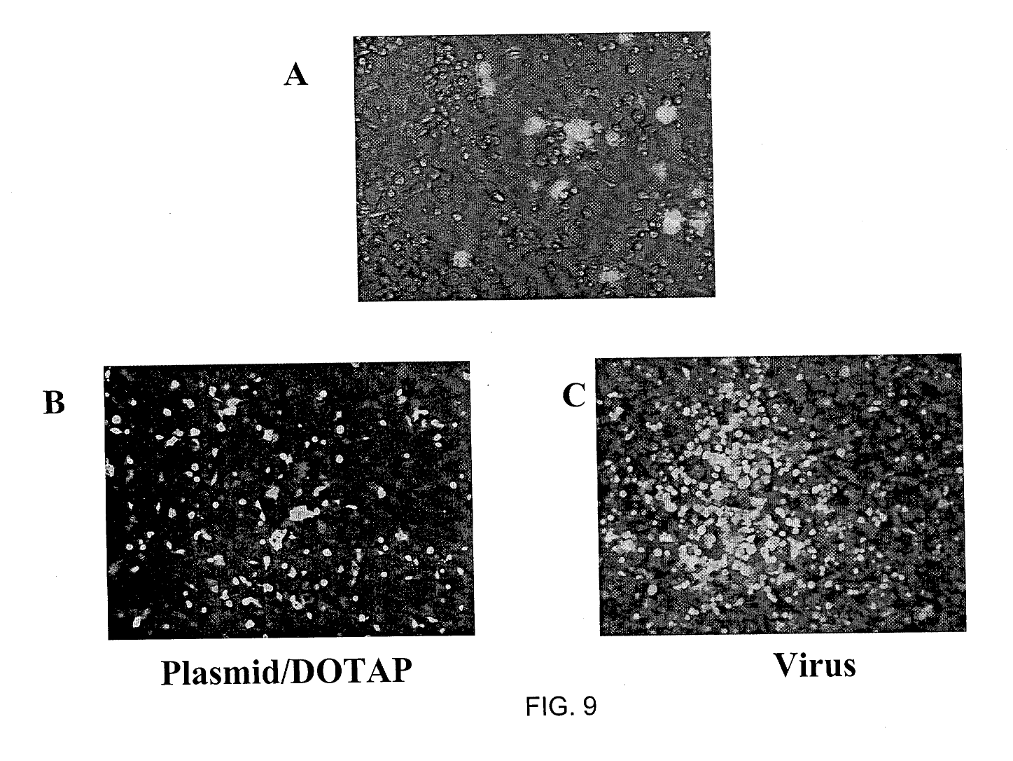

Ad5#5 bfp plasmid, PFG140 plamid and dl1520 viral DNA were utilized to construct the circular plasmid. FIG. 8 shows the details of the cloning steps. DNA digestion with Hind III enzymes showed both dl1520 viral DNA and the circular dl1520 DNA had the intended deletion in E1b region with retention of the remainder dl1520 genome.

### Example 12

In vitro Transfer Efficacy of DOTAP

DOTAP DNA transfer efficacy was evaluated by treating H1299 cells with DOTAP complexed to a plasmid containing the GFP gene. Ad5-GFP was used as a control. Twenty-four hours post-treatment, positive green cells were visualized with a fluorescent microscope. The viral gene transfer was high on these CAR positive cells. The plasmid-GFP transfer was efficient but, as expected, was lower than what was obtained with an Ad5-GFP virus (FIGS. 9A through 9C) 20-30% of the cells were transfected. transfection with plasmid.

### Example 13

dl1520 Lyse p53 Null Cells

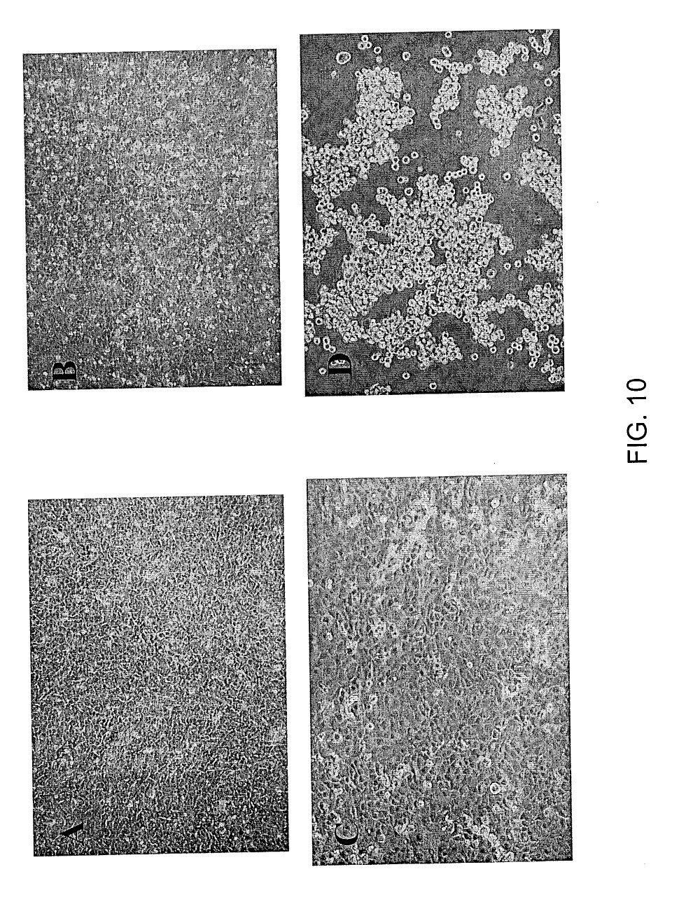

The p53 abnormal cells, H1299 and T24 were transduced in vitro with dl1520 (10³ vp/cell), and all cells were maintained until complete CPE was observed. FIG. 10 shows that only the H1299 cells were lysed. T24 cells, which comprise a p53 mutant having an in-frame deletion of tyrosine 126, remained alive weeks after the experiment was terminated. This result is explained by the very low expression of CAR by those cells; the virus poorly binds to the cells, resulting in a very low transduction efficiency.

### Example 14

dl1520 DNA Lyse Tumoral Cell Lines

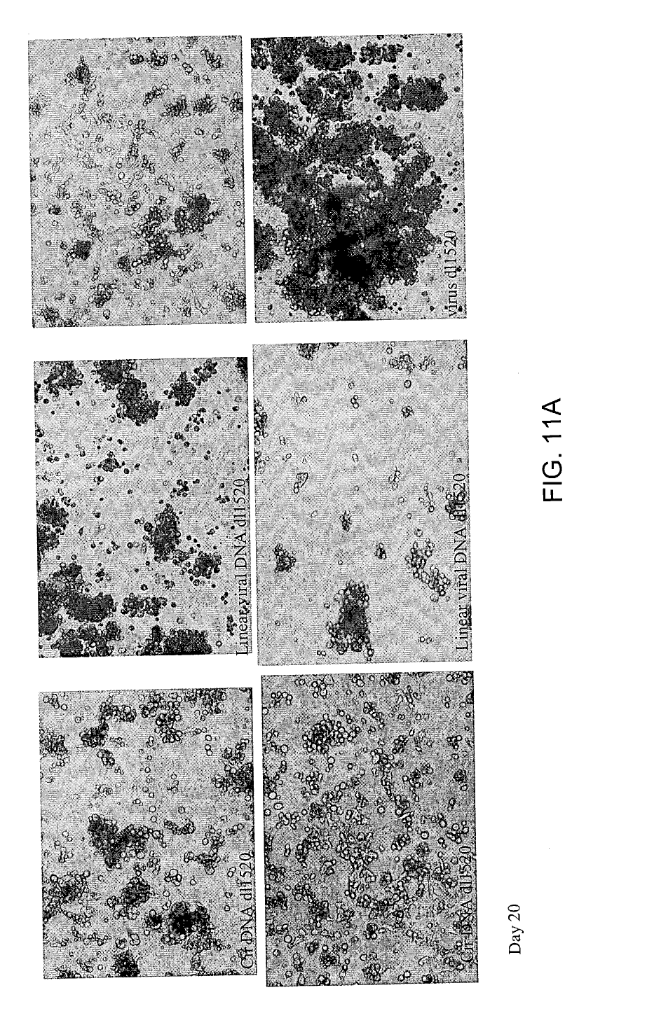

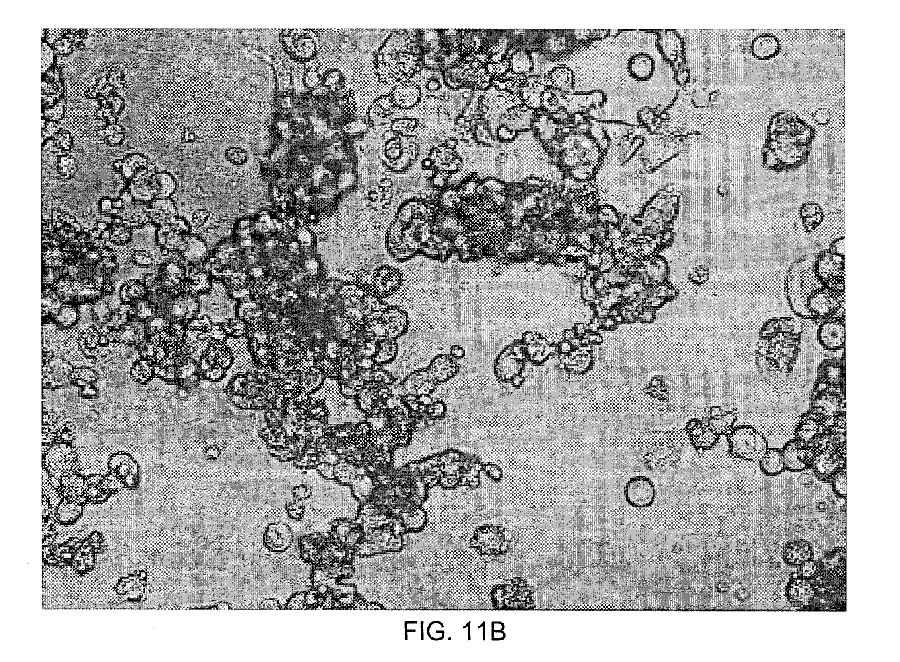

To investigate if the linear and circular dl1520 could lyse tumoral p53-null cells, H1299 cells were transduced with those vectors. As a control, 293 cells were used. The CPE effect was observed on 293 cells treated with both virus and DNA (linear and circular), suggesting that the viruses produced were infectious. FIG. 11A represents the results for the CPE assay. As expected, virus dl1520 induced lysis of H1299, suggesting a replication of the virus in the p53-null cells. The linear and circular dl1520 DNA induced the lysis of H1299, confirming that they were replicative and that the viruses produced were infectious. The E1 deleted Ad-GFP did not lyse H1299 cells. FIG. 11B is a higher magnification of H1299 cells lysis following circular dl1520 DNA transfection.

### Example 15

Production of Infectious Virus with Circular dl1520

To evaluate the titer of the virus produced by the circular and linear dl1520 DNA, H1299 cells were transduced with DOTAP/viral dl1520, DOTAP/circular dl1520, and dl1520 virus, the cells were harvested when almost all the cells were lysed. The infectious viral particles produced were titrated by OD₂₆₀. H1299 produced infectious virus following infection with either viral linear dl1520 DNA, or dl1520 circular DNA. As expected, the titer was higher with virus dl1520 (Table 5), and the lytic effect appeared first in wells containing cells treated with the virus than in the wells containing cells treated with linear virus, and finally in wells containing cells treated with the circular virus.

TABLE 5
Titration of dl1520 produced by H1299 cells at day 20

| Type of dL1520 | 10¹⁰ vp/ml (±std) |
|---|---|
| Virus | 49 (±0.0001) |
| Circular DNA | 8.5 (±0.026) |

Even if the lytic effect was delayed, all of the cells from each wells were lysed by the virus. The number of infectious particles rapidly increases with the time after a long phase, presumably representing the time required for initial viral protein production and virus assembly from the plasmid DNA, after that phase the viral replication rate is equal to what observe after a virus infection of H1299.The difference in titer only reflect a delay of virus production (all of the cells were harvested the same day). The same titer was obtained in all conditions when the cells were harvested sequentially when the cells were lysed at 100%.

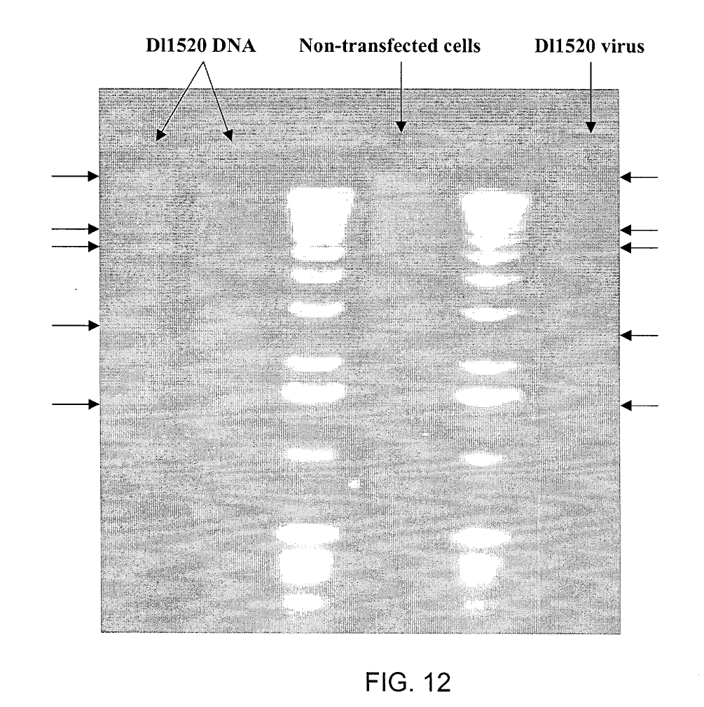

To ensure that the viruses produced by DNA transfer were the same as the one produced after virus infection, an enzymatic digestion of those viruses produced in H1299 cells was performed using Xho I. The results visualized on a gel (FIG. 12) showed that the viruses produced after infection with dl1520 viruses or dl1520 DNA transfer gave the same number of bands. Those bands were also of the same size implying that the viruses were identical.

### Example 16

Neutralizing Antibodies do not Affect Liposome/dl1520

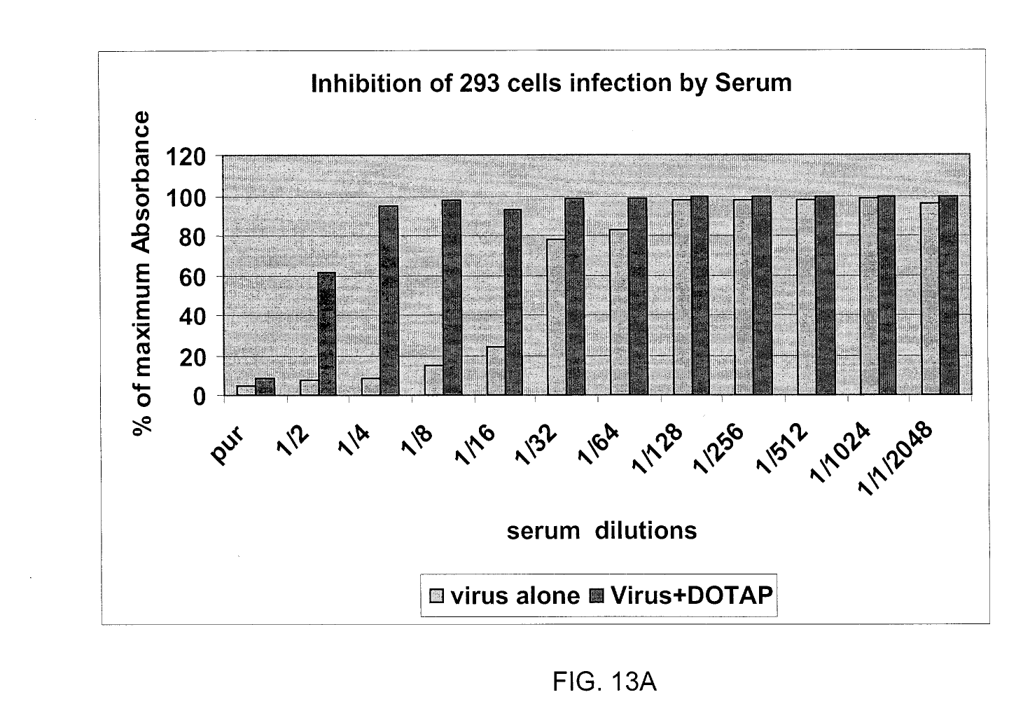

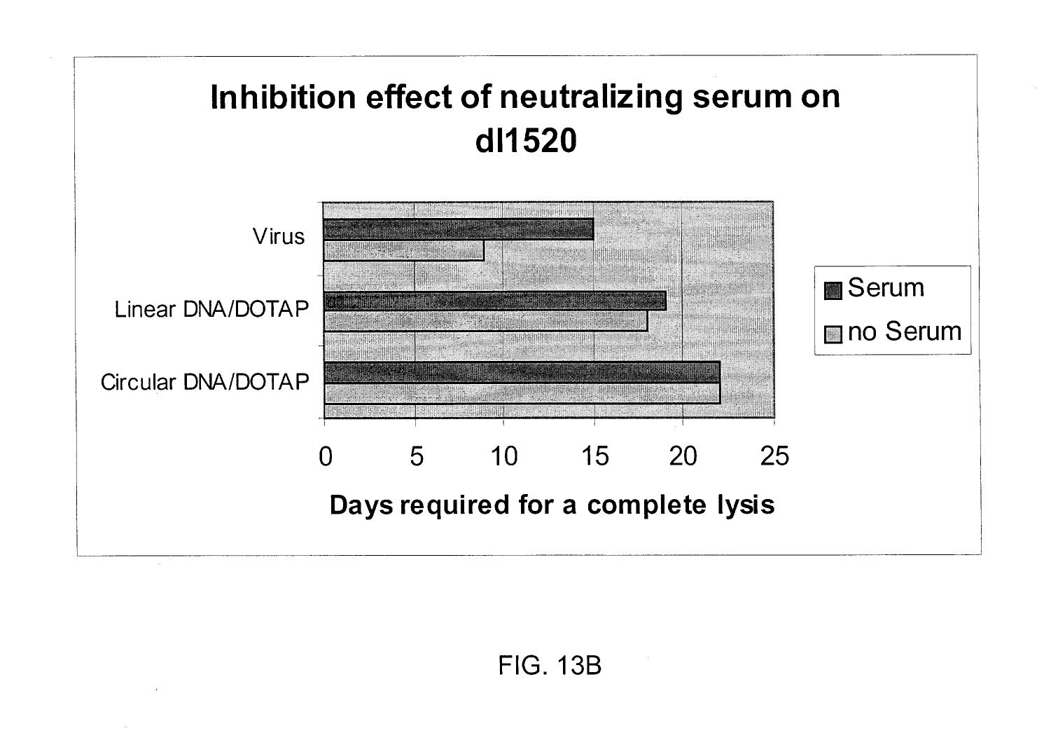

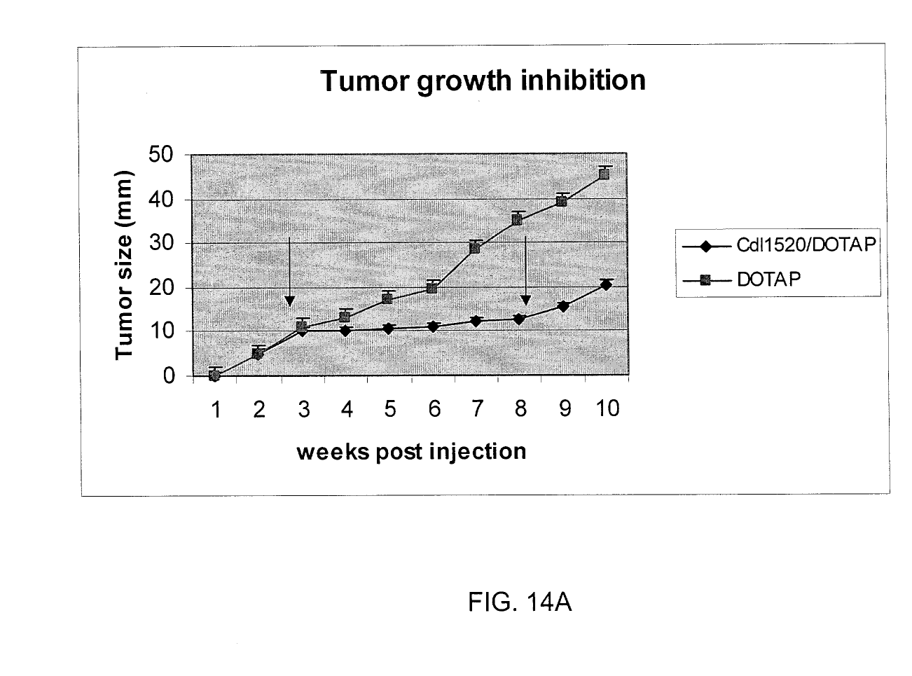

Ad5 have been reported to be neutralized by serum in pre-immunized host. The protective effect of DOTAP in the presence of Ad5 neutralizing serum (incubated 1 h at 37° C.) was evaluated. The H1299 cells were infected with dl1520 virus or DNA coated with liposome and exposed or not exposed to the serum. As expected, the infection of the free virus was significantly inhibited by the pre-incubation with serum (FIGS. 13A and 13B). The plasmid and linear DNA coated with liposome was not significantly affected by the presence of serum.

### Example 17

Tumor Growth Inhibition Effect of Circular dl1520

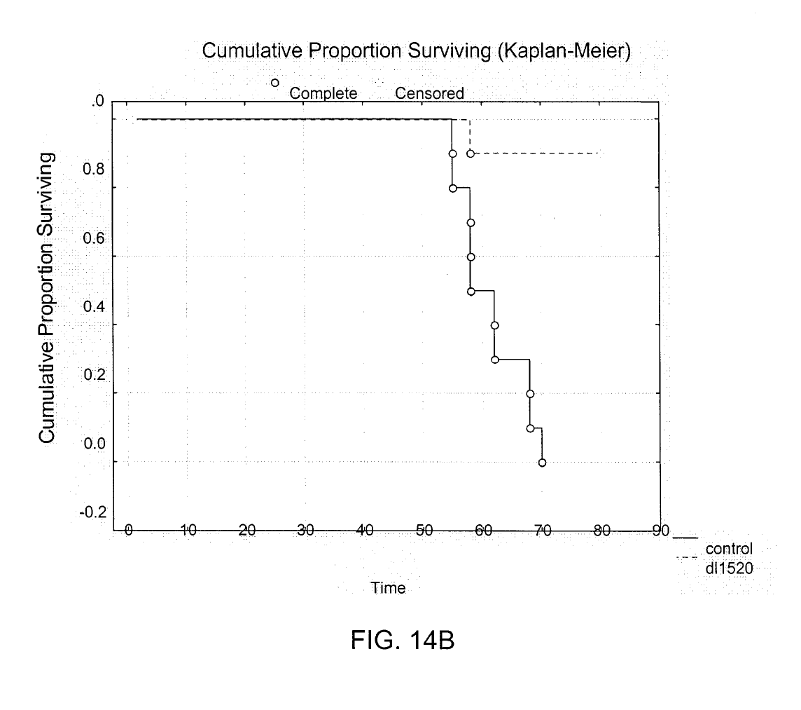

The antitumor effects of circular dl1520 DNA were evaluated in human xenograft models using H1299 cells (p53-null). SCID mice or Nude mice (n=10) were injected sc with the tumor cells (10⁷ cell/mouse). dl1520 was administered at doses ranging from 50-100μg per injection to mice with tumors with diameters of about 5 mm (first arrow, at week 3), whereas randomly allocated control mice received injections with liposome alone or Ad-GFP plasmid/liposome. As observed with dl1520 virus, intratumoral injection of DOTAP/circular dl1520 into the p53-null tumor stopped the tumor growth (FIG. 14A). No growth retardation of the tumor was observed in liposome alone or Ad-GFP plasmid-treated tumors. As others have reported (Vollmer et al., 1999; Nemunaitis et al., 2000), total tumor regression was a very rare event, the tumors were refractive when the treatment ceased (second arrow, at week 8). However, mice injected with DOTAP/circular dl1520 survive longer than control mice (FIG. 14B). At day 50 control mice were very sick and started to die shortly after. By day 60, all the control mice were dead. In the group treated with cdl1520, only one mouse died at day 58; all of the other mice survived until they had to be sacrificed.

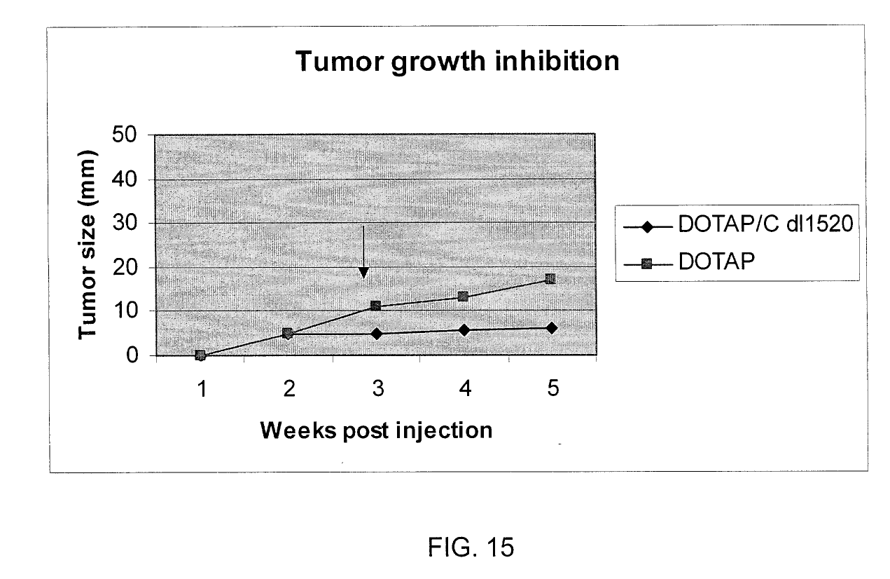

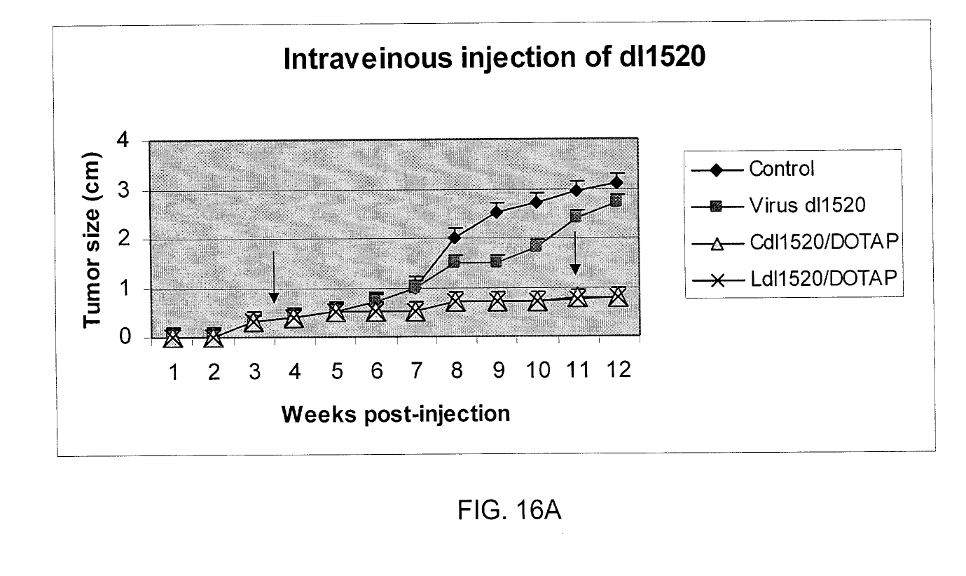

In a second model of mice treatment, the same tumoral cells were transfected in vitro with dl1520 circular DNA and injected i.t in the back of the SCID mice. After tumor growth, the tumor was injected as previously described. The inhibition of the tumor proliferation was obtained “faster” than with no, pretreatment with dl1520 plasmid injection (FIG. 15), implying that the virus started replicating earlier in the tumor by consequence this induced a difference in rate of virus production or spreading in vivo. But again, no tumor regression was observed, and the tumors grew back upon cessation of the injection of dl1520 to the mice.

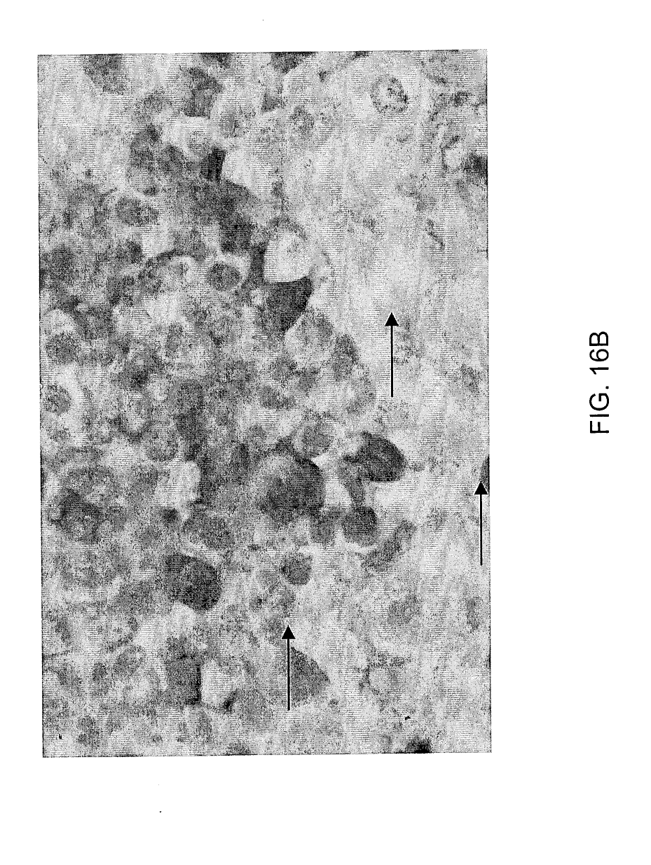

In order to show the efficacy of this treatment when systemic injection had to be done, dl1520 virus and DNA pre-incubated with human neutralizing serum was injected in the tail vein of the tumor-bearing mice. Mice were injected every 2 days with dl1520 (both virus and DNA). Treatment started at week 4 and was stopped at week 11. To avoid having a low efficiency of the virus due to a big tumor burden, less cells (3-5×10⁶/mouse) were injected than in previous experiments. A weaker efficiency of the dl1520 virus itself compared to the therapeutic effect obtained with dl1520 DNA was observed. The circular and linear forms gave slightly similar results; they both strongly delay the tumor growth (FIG. 16A). At the end of the treatment, mice injected with the virus had tumors growing back to a size similar to that observed for the tumor of the non-treated group. After 12 weeks, the tumors slowly resumed growing but never reached the size of the tumor in control or virus treated mice. The detection of adenovirus by immunocytochemistry confirmed that the virus replicates in the tumor (FIG. 16B). That is, in specific embodiments, the virus injected is neutralized by the antibodies, the plasmid injected reach the cells and is internalized, then viruses are produced from that cell (with the plasmid); this new virus will travel from cell to cell and “sees” almost no serum since the propagation is done to the neighboring immediate cells and not through the blood.

### Example 18

Significance of the Present Invention

The present invention demonstrates that complexing adenovectors with bilamellar liposomes serves to alter their target cell range ex vivo and to protect them from human neutralizing antibody ex vivo and in vivo. Previous reports demonstrated that adenovectors were unable to infect cells lacking CAR and integrins (Grubb et al., 1994). In contrast, liposomes have been shown to be receptor independent for cell entry (Friend et al., 1996). Once the negatively-charged adenovectors are non-covalently complexed to positively charged liposomes (DOTAP:cholesterol), adenoviruses binding to the negatively charged cell membrane independently of any receptor/integrin interaction (Fasbender et al., 1997), probably following an electrostatic interaction with the cell's sialic acid residues. Heparan sulfate has also been shown to play a role in this binding. Once bound, Ad/liposome complexes cross the membrane by endocytosis, a process whose efficiency depends on the charge density of the complex, the time of incubation with the cells (Zabner et al., 1995) and to a lesser extent, the complex size (Meunier-Durmort et al., 1997). The intracellular barriers are overcome by the adenoviral protein-dependent functions (Greber et al., 1993), releasing the complex by lysis of the endosome, (Meunier-Durmort et al., 1997) and transporting the DNA into the nucleus for a high level of gene expression.

The above effects were readily observed in the Examples of the present invention, with high GFP expression obtained in cells resistant to Ad 5 infection due to their lack of CAR/integrin expression (CHO, NIH 3T3). These results are consistent with earlier findings using conventional cationic lipids (Fasbender et al., 1997; Qiu et al., 1998). Of note, limited enhancement of transgene expression in cells already permissive for Ad 5 infection was observed, at low vp:cell ratios (Table 1). Hence, this strategy allows efficient gene transfer and expression even in tissues lacking CAR/integrins. By reducing the vector load required to transduce relatively resistant cells, the technique limits toxicity.

A more immediately important feature of the lipid described herein the Examples is its ability to shield adenovector from otherwise neutralizing humoral immune responses, both human and mouse. Most lipids incorporating DOPE (dioleoylphosphatidylethanolamine) have been described as semi-fused or loose structures at low DNA:lipid ratio and short co-incubation time. Other lipids like DOTMA (N-(2,3-dioleoyloxypropyl)-N,N-trimethylammonium chloride) form a spaghetti (liposomes) and meatball (single lipid bi-layer) structure at high ratio and after prolonged incubation. Other authors have described lipid encapsulation of DNA in a unilamellar structure. DOTAP (dimethyldioctacylammonium propane) and DDAB:DOPE combine with DNA to form a multi-lamellar structure at low DNA:lipid ratios (Gustafsson et al., 1995). Therefore, at high DNA:lipid ratios, free DNA remains outside the lipoplex. In contrast, Smyth Templeton et al described how DOTAP:chol forms a bilamellar structure that completely encapsulates DNA (Templeton et al., 1997). Bilamellar structures consist of two bilayers or lamella, as previously described (Templeton et al., 1997). These bilamellar invaginated structures are distinct from multilamellar structures previously reported (Gustafsson et al., 1995), which are multi-layered consisting of more than only two bilayers. Most of these studies used plasmid DNA, but the present invention shows that DOTAP:chol can also fully encapsulate intact adenovectors, as confirmed by electron micrographs.

The DOTAP:chol/AV complexes described herein are evidently stable in the presence of human serum (Chirmule et al., 1999; Zhang et al., 2001) and are also functionally protected from human and murine neutralizing antibodies. In vitro assays, performed from 0-24 hours at 37° C., demonstrated that complexes are stable at physiological concentrations of serum as found in the bloodstream. These complexes are stable in vivo after intravenous injections and produce high levels of gene expression in all organs assayed (Templeton et al., 1997).

Even in the presence of high concentrations of human adenovirus neutralizing serum, 293 cells were readily infected by DOTAP:chol-protected adenovector while uncoated adenovirus and conventional lipofectamine coated virus gave a low transfection level (Meunier-durmort et al., 1997). Similar in vitro protective effects from immune mouse serum have been reported using PEG, (Croyle et al., 2001), PLGA (Beer et al., 1998), TMAG:DLPC:DOPE (Natsume et al., 2000), and pHMA (Fisher et al., 2001) treatment of vectors. However, it is not clear whether these alternative approaches to advector concealment allow entry into adenovirus resistant (CAR negative) cell lines, or protect against human as well as murine anti-adenovector antibody. Nor is it known if such alternative approaches reduce the ability of the treated virus to release inflammatory cytokines as demonstrated with the DOTAP-chol/AV complexes used here (FIG. 7).

Liposome mediated protection from external antibodies needs to be particularly effective, since liposomes themselves are often effective immune adjuvants for the proteins they carry. This effect is likely associated with their ability to be taken up by antigen presenting cells. Mice were injected with a vector containing a marker gene or a potentially therapeutic gene (hAAT) and then re-injected one month later to evaluate the protective activity of the liposomes against neutralization by antibodies present in the serum of these mice. In the Ad-lac-Z model, mice produced neutralizing anti-adenovirus antibodies and the titers were substantially higher in mice injected with liposome/Ad complexes than the virus alone. Nonetheless, in this model a higher expression of the transgene with DOTAP:chol/adenovirus injection was obtained, both on first and second injection, than with the naked virus. Hence, in specific embodiments DOTAP:chol augments humoral immune responses, but protects the virus from neutralization. Because the humoral anti-adenovirus immune response generated in humans may differ from mice, human neutralizing serum was also used to obtain a more accurate assessment of the likely protective activity of the DOTAP:chol in the clinical setting. Mice injected with liposomes pre-incubated in neutralizing-human serum still showed a high level of transgene expression in lung and liver. Similar results were obtained with hAAT, in which pre-incubation with human neutralizing serum markedly reduced the level of hAAT expression from naked but not liposome coated advector. While it may be possible to obtain infection with naked virus in the presence of neutralizing antibody (as shown in monkeys (Nunes et al., 1999) and some humans (Gahery-Segard et al., 1998)) the inevitable reduction in infectivity requires that a larger dose be administered for a given effect (Chirmule et al., 1999). This in turn favors development of the acute toxicities that may occur with such devastating consequences in humans injected with Ad vectors (Zhang et al., 2001). An inflammatory response involving release of IL6 appears to be an important initiating factor in this acute toxicity (Scheule et al., 1997; Zxengeller et al., 2000), and the observation that the naked virus induces substantially greater IL6 than encapsulated virus may be another advantage for encapsulated vector.

Hence, the results show that adenovirus can be completely encapsulated in bilamellar cationic liposomes. Encapsulation changes the target cell range of the vector, while leaving unimpeded its ability to produce high level gene expression. The coated adenovectors, while immunogenic, are nonetheless protected from human neutralizing antibodies ex vivo and in vivo (in mice), and so can be readily re-administered. These liposomal-advector chimeras may therefore be valuable in applications in which repeat administration of the vectors is desirable and where the acute inflammatory response needs to be minimized.

Replicating adenovirus dl1520 lyses p53 abnormal tumors and is a useful therapeutic approach for a large number of tumors. The first results of clinical trial using dl1520 showed a significant selective antitumoral effect of dl1520 (Goodrum et al., 1998; Rothmann et al., 1998; Tumell et al., 1999; Bischoff et al., 1996; Kim, 2001; Heise et al., 2000; Nemunaitis et al., 2000; Nemunaitis et al., 2001), with no damage on normal surrounding tissues. Unfortunately, as for all the Ad5, its effect is limited by the necessity to have the CAR receptor and the integrins (αVβ5, αVβ3) expressed at the target cell surface and by the anti-adenovirus immune response generated by a previous exposition to the virus. To overcome those limits, high MOI are often used, raising many safety issues for patients. The present Examples described the advantage of using a dl1520 DNA over the viral form of dl1520 to increase the safety. The present invention uses cationic liposome to vehicle dl1520 to increase the efficacy of delivery of both dl1520 virus and DNA to overcome this disadvantage.

Adenovirus genome comprises a linear double stranded DNA molecule characterized by two inverted terminal repeat sequences. This linear DNA is considered to be a template to initiate the replication by either end of the molecule. Ruben et al. (1983) showed that a circular adenovirus (Ad5) genome is present in infected cells. This circular DNA is never found in virion and is probably due to a head to tail formation of the DNA. Graham cloned a plasmid Ad5 circle called pFG140 replicating in Escherichia coli. (Graham et al., 1989; Graham, 1984). In 293 cells, this plasmid is able to generate infectious viruses with an efficiency comparable to what was obtained with Ad5 virus. Based on pFG140 and dl1520 DNA, an infectious circular dl1520 plasmid DNA was generated. First, the present inventors demonstrated that cationic liposome (DOTAP:chol) could be used to efficiently transfer Ad5-plasmid to target cells. The injection of the Ad-plasmid alone would only transform a non-significative percentage of cells. The use of other liposomes would result in a lower efficiency; since these liposomes have a semi-fused, loose, or spaghetti and meatball structure that let the DNA partially or completely outside the lipolex. Moreover, DOTAP:chol was also shown to be stable at 37 degrees in presence of serum, making it even more suitable for cell transformation with plasmid.

Second, it was shown that DOTAP efficiently transfer the dl1520 construct into target cells, since 293 cells tranfected with the DNA were lysed. Finally, it was shown that the transferred circular dl1520 DNA like dl1520 virus is infectious and replicates in p53-null tumor cells. The H1299 cells were efficiently lysed and the viruses produced were infectious, the enzymatic analysis of the viruses produced showed that the Xho I pattern obtained was identical to that obtained with the parental dl1520 virus. Also, cationic liposome complexed to dl1520 virus increased its efficiency. The low efficiency of free dl1520 in clinical protocol could be explained by the sensitivity of adenovirus to neutralizing antibodies, especially in patients already exposed to the virus (Ganly et al., 2000; Freytag et al., 1998; Mulvihill et al., 2001; Nemunaitis et al., 2001). In contrast, cationic liposome also allows transduction in the presence of serum by forming a protective coat around the DNA and the virus. In vitro, it was shown that adenovirus-neutralizing serum was ineffective on circular or linear dl1520/DOTAP and dl1520virus/DOTAP transduction compared to its effect on free dl1520 virus. In vivo, mice bearing H1299 tumor injected intratumorally with circular or linear dl1520/DOTAP:chol had smaller tumors and lived longer than non vaccinated control mice. When the treatment was injected intravenously, there was a superior protection of the mice with the DOTAP:chol/dl1520 DNA than for the mice injected with the free virus. Nemunmatis described in a head and neck tumor a therapeutic effect of dl1520 virus that decreased at the end of the treatment (Nemunaitis et al., 2000). However, dl1520 was shown to be efficient only when associated to other therapy such as radiotherapy, 5-FU, or cisplatin (Heise et al., 2000; Heise et al., 1997; Vollmer et al., 1999; Lamont et al., 2000; Khuri et al., 2000). Similarly, it was observed that the inhibition of the tumor growth ceased with circular dl1520/DOTAP treatment. Furthermore, as observed with dl1520 virus, the circular dl1520/DOTAP could inhibit the tumor growth but never induce tumor regression when injected as a single drug. Several observations could explain the reported limited success of dl1520 virus. The p53 status of the cells, but also the p53 heterogeneity of the tumor in vivo, should also be considered (Mirchandani et al., 1995). The tumor burden at the time of the treatment is an important factor to be considered, (Heise et al., 2000) as well as the tumor structure and the extracellular martrix barrier (Kuppen et al., 2001; Bilbao et al., 2000). The cellular immune response generated by the viral protein can also activate macrophages and CTL to eliminate the infected cells and limit the replication of the virus (Geutskens et al., 2000). At this stage of the response, the tumor burden can affect the efficacy of this strategy, since th is viral specific response can induce a tumor-specific response and increase a dl1520 therapeutic effect. Nevertheless, a better inhibition was obtained with dl1520 DNA than with the dl1520 virus. A longer effect of the treatment was observed after the injections were stopped, since the tumor grew back slower in the group receiving DNA than in the group of mice injected with dl1520 virus. This observation could be explained by the fact that circular dl1520 is not limited like viral dl1520 by the level of CAR expression at the cell surface. The DNA is entirely encapsulated by a bilamellar liposomal envelope and crosses the cell membrane in a passive, CAR independent way. dl1520 have been mainly used for solid tumor treatment because of the needle limitation access. Injected systemically into mice, dl1520 is trapped in the liver (Heise et al., 1999). dl1520 is injected at high MOI to overcome this point. Even if the toxicity in the liver was moderated in the only human study reported (Nemunaitis et al., 2001). DOTAP:chol bilamellar liposome was reported by others and the present inventors to be non-toxic and to have efficiently delivered plasmid DNA to many tissues and organs, including lung and liver parenchyme when injected in systemic. This large biodistribution is another advantage of the use of the plasmid over the virus dl1520. Because DOTAP:chol encapsulates the plasmid, it not only delays the immune response but also protects the genetic material from the host immune response. This effect leads to a larger amount of DNA available to transform the target cell and a consequently higher/longer expression of the genetic material transferred. This new strategy of dl1520 delivery will improve efficiency and safety of the dl1520 anti-tumor therapy.

References

All patents and publications mentioned in the specification are indicative of the level of those skilled in the art to which the invention pertains. All patents and publications are herein incorporated by reference to the same extent as if each individual publication was specifically and individually indicated to be incorporated by reference.

Patents

U.S. Pat. No.4,728,578

U.S. Pat. No.4,728,575

U.S. Pat. No.4,737,323

U.S. Pat. No.4,533,254

U.S. Pat. No.4,162,282

U.S. Pat. No.4,310,505

U.S. Pat. No.4,921,706

U.S. Pat. No.5,401,511

U.S. Pat. No.5,432,260

U.S. Pat. No.5,603,872

U.S. Pat. No.5,635,380

U.S. Pat. No.5,786,214

U.S. Pat. No.5,849,718

U.S. Pat. No.5,871,727

U.S. Pat. No.5,879,703

U.S. Pat. No.5,899,155

U.S. Pat. No.5,908,635

U.S. Pat. No.5,928,944

U.S. Pat. No.5,939,277

U.S. Pat. No. 6,107,090

U.S. Pat. No. 6,110,490

U.S. Pat. No. 6,133,243

U.S. Pat. No. 6,156,497

U.S. Pat. No. 6,228,646

U.S. Pat. No. 6,413,544

Publications

Armentano, D., et al., Effect of the E4 region on the persistence of transgene expression from adenovirus vectors. J Virol, 1997. 71(3): p. 2408-2416.

Bangham, et al, “Diffusion of univalent Ions across the Lamellae of Swollen Phospholipids” J. Mol. Biol., 13:238-252, 1965.

Barker D D, Berk A J. Adenovirus proteins from both E1B reading frames are required for transformation of rodent cells by viral infection and DNA transfection. Virology. January 1987;156(1):107-21.

Beer, S. J., et al., Poly (lactic-glycolic) acid copolymer encapsulation of recombinant adenovirus reduces immunogenicity in vivo. Gene Ther, 1998. 5(6): p. 740-706.

Bellon, G., et al., Aerosol administration of a recombinant adenovirus expressing CFTR to cystic fibrosis patients: a phase I clinical trial. Hum Gene Ther, 1997. 8(1): p. 15-25.

Bilbao, R., M. Bustos, et al. (2000). “A blood-tumor barrier limits gene transfer to experimental liver cancer: the effect of vasoactive compounds.” Gene Ther 7(21): 1824-32.

Bischoff, J. R., D. H. Kirn, et al. (1996). “An adenovirus mutant that replicates selectively in p53-deficient human tumor cells.” Science 274(5286): 373-6.

Brody, S. L., et al., Acute responses of non-human primates to airway delivery of an adenovirus vector containing the human cystic fibrosis transmembrane conductance regulator cDNA. Hum Gene Ther, 1994. 5(7): p. 821-836.

Buttgereit, et al., “Efficient gene transfer into lymphoma cells using adenoviral vectors combined with lipofection” Cancer Gene Therapy, Vol. 7, No. 8, 2000: pp 1145-1155.

Caplen, N. J., et al., Liposome-mediated CFTR gene transfer to the nasal epithelium of patients with cystic fibrosis. Nat Med, 1995. 1(1): p. 39-46.

Chirmule, N., et al., Immune responses to adenovirus and adeno-associated virus in humans. Gene Ther, 1999. 6(9): p. 1574-1583.

Croyle, M. A., et al., “Stealth” adenoviruses blunt cell-mediated and humoral immune responses against the virus and allow for significant gene expression upon readministration in the lung. J Virol, 2001. 75(10): p. 4792-4801.

Crystal, R. G., et al., Administration of an adenovirus containing the human CFTR cDNA to the respiratory tract of individuals with cystic fibrosis. Nat Genet, 1994. 8(1): p. 42-51.

Deamer and P. Uster, “Liposome Preparation: Methods and Mechanisms,” in Liposomes (M. Ostro, ed.), Marcel Dekker, Inc., New York (1983), pp. 27-52.

DRUG CARRIERS IN BIOLOGY AND MEDICINE, G. Gregoriadis ed. (1979) pp. 287-341.

Dubochet, J., et al., Cryo-electron microscopy of vitrified specimens. Q Rev Biophys, 1988. 21(2): p. 129-228.

Fasbender, A., et al., Complexes of adenovirus with polycationic polymers and cationic lipids increase the efficiency of gene transfer in vitro and in vivo. J Biol Chem, 1997. 272(10): p. 6479-6489.

Fisher, K. D., et al., Polymer-coated adenovirus permits efficient retargeting and evades neutralising antibodies. Gene Ther, 2001. 8(5): p. 341-348.

Fraley and Fomari Kaplan, “Entrapment of a bacterial plasmid in phospholipid vesicles:potential for gene transfer,” Proc. Nat'l. Acad. Sci. USA 76:3348-3352, 1979.

Friend, D. S., D. Papahadjopoulos, and R. J. Debs, Endocytosis and intracellular processing accompanying transfection mediated by cationic liposomes. Biochim Biophys Acta, 1996. 1278(1): p. 41-50.

Freytag, S. O., K. R. Rogulski, et al. (1998). “A novel three-pronged approach to kill cancer cells selectively: concomitant viral, double suicide gene, and radiotherapy.” Hum Gene Ther 9(9): 1323-33.

Gahery-Segard, H., et al., Humoral immune response to the capsid components of recombinant adenoviruses: routes of immunization modulate virus-induced Ig subclass shifts. Eur J Immunol, 1997. 27(3): p. 653-659.

Gahery-Segard, H., et al., Immune response to recombinant capsid proteins of adenovirus in humans: antifiber and anti-penton base antibodies have a synergistic effect on neutralizing activity. J Virol, 1998. 72(3): p. 2388-2397.

Gahery-Segard, H., et al., Phase I trial of recombinant adenovirus gene transfer in lung cancer. Longitudinal study of the immune responses to transgene and viral products. J Clin Invest, 1997. 100(9): p. 2218-2226.

Ganly, I., D. Kim, et al. (2000). “A phase I study of Onyx-015, an E1B attenuated adenovirus, administered intratumorally to patients with recurrent head and neck cancer.” Clin Cancer Res 6(3): 798-806.

Ghosh and Bachhawat, “Targeting of liposomes to hepatocytes,” In: Wu G. Wu C ed., Liver diseases, targeted diagnosis and therapy using specific receptors and ligands, New York: Marel Dekker, pp. 87-104, 1991.

Goodrum, F. D. and D. A. Ornelles (1998). “p53 status does not determine outcome of E1B 55-kilodalton mutant adenovirus lytic infection.” J Virol 72(12): 9479-90.

Graham, F. L. (1984). “Covalently closed circles of human adenovirus DNA are infectious.” Embo J 3(12): 2917-22.

Graham, F. L., J. Rudy, et al. (1989). “Infectious circular DNA of human adenovirus type 5: regeneration of viral DNA termini from molecules lacking terminal sequences.” Embo J 8(7): 2077-85.

Greber, U. F., et al., Stepwise dismantling of adenovirus 2 during entry into cells. Cell, 1993. 75(3): p. 477-486.

Gregoriadis G. and Davis C. “Stability of liposomes in vivo and in vitro is promoted by their cholesterol content and the presence of blood cells,” Biochem Biophys Res Commun., 89(4):1287-1293, 1979.

Gregoriadis, DRUG CARRIERS IN BIOLOGY AND MEDICINE, G. Gregoriadis (ed.), 1979, pp. 287-341.

Grubb, B. R., et al., Inefficient gene transfer by adenovirus vector to cystic fibrosis airway epithelia of mice and humans. Nature, 1994. 371(6500): p. 802-806.

Geutskens, S. B., M. M. van der Eb, et al. (2000). “Recombinant adenoviral vectors have adjuvant activity and stimulate T cell responses against tumor cells.” Gene Ther 7(16): 1410-6.

Gustafsson, J., et al., Complexes between cationic liposomes and DNA visualized by cryo-TEM. Biochim Biophys Acta, 1995. 1235(2): p. 305-312.

Harada J N, Berk A J. p53-Independent and -dependent requirements for E1B-55K in adenovirus type 5 replication. J Virol. July 1999;73(7):5333-44.

Heise, C., I. Ganly, et al. (2000). “Efficacy of a replication-selective adenovirus against ovarian carcinomatosis is dependent on tumor burden, viral replication and p53 status.” Gene Ther 7(22): 1925-9.

Heise, C., M. Lemmon, et al. (2000). “Efficacy with a replication-selective adenovirus plus cisplatin-based chemotherapy: dependence on sequencing but not p53 functional status or route of administration.” Clin Cancer Res 6(12): 4908-14.

Heise, C., A. Sampson-Johannes, et al. (1997). “ONYX-015, an E1B gene-attenuated adenovirus, causes tumor-specific cytolysis and antitumoral efficacy that can be augmented by standard chemotherapeutic agents.” Nat Med 3(6): 639-45.

Heise, C. C., A. M. Williams, et al. (1999). “Intravenous administration of ONYX-015, a selectively replicating adenovirus, induces antitumoral efficacy.” Cancer Res 59(11): 2623-8.

Ilan, Y., et al., Insertion of the adenoviral E3 region into a recombinant viral vector prevents antiviral humoral and cellular immune responses and permits long-term gene expression. Proc Natl Acad Sci USA, 1997. 94(6): p. 2587-2592.

Jooss, K., H. C. Ertl, and J. M. Wilson, Cytotoxic T-lymphocyte target proteins and their major histocompatibility complex class I restriction in response to adenovirus vectors delivered to mouse liver. J Virol, 1998. 72(4): p. 2945-2954.

Jooss, K., L. A. Turka, and J. M. Wilson, Blunting of immune responses to adenoviral vectors in mouse liver and lung with CTLA4Ig. Gene Ther, 1998. 5(3): p. 309-319.

Kaplan, J. M. and A. E. Smith, Transient immunosuppression with deoxyspergualin improves longevity of transgene expression and ability to readminister adenoviral vector to the mouse lung. Hum Gene Ther, 1997. 8(9): p. 1095-1104.

Kaplan, J. M., et al., Humoral and cellular immune responses of nonhuman primates to long-term repeated lung exposure to Ad2/CFTR-2. Gene Ther, 1996. 3(2): p. 117-127.

Kaneda et al, “Increased expression of DNA cointroduced with nuclear protein in adult rat liver,” Science, 243:375-378, 1989.

Kaneda et al., “Introduction and expression of the human insulin gene in adult rat liver,” J Biol Chem., 264(21):12126-12129, 1989.

Kato et al., “Expression of hepatitis B virus surface antigen in adult rat liver. Co-introduction of DNA and nuclear protein by a simplified liposome method,” J Biol Chem., 266(6):3361-3364, 1991.

Kay, M. A., et al., Expression of human alpha 1-antitrypsin in dogs after autologous transplantation of retroviral transduced hepatocytes. Proc Natl Acad Sci USA, 1992. 89(1): p. 89-93.

Khuri, F. R., J. Nemunaitis, et al. (2000). “a controlled trial of intratumoral ONYX-015, a selectively-replicating adenovirus, in combination with cisplatin and 5-fluorouracil in patients with recurrent head and neck cancer.” Nat Med 6(8): 879-85.

Kim D, Hermiston T, McCormick F. ONYX-015: clinical data are encouraging. Nat Med. December 1998;4(12):1341-2. Lee, S. G. et al., “Enhancement of adenoviral transduction with polycationic liposomes in vivo,” Cancer Gene Therapy, Vol 7, Nov. 10, 2000: pp.1329-1335.

Kim D. Oncolytic virotherapy for cancer with the adenovirus dl1520 (Onyx-015): results of phase I and II trials. Expert Opin Biol Ther. May 2001;1(3):525-38. Review.

Kim D. Clinical research results with dl1520 (Onyx-015), a replication-selective adenovirus for the treatment of cancer: what have we learned? Gene Ther. January 2001;8(2):89-98.

Kuppen, P. J., M. M. van der Eb, et al. (2001). “Tumor structure and extracellular matrix as a possible barrier for therapeutic approaches using immune cells or adenoviruses in colorectal cancer.” Histochem Cell Biol 115(1): 67-72.

Lamont, J. P., J. Nemunaitis, et al. (2000). “A prospective phase II trial of ONYX-015 adenovirus and chemotherapy in recurrent squamous cell carcinoma of the head and neck (the Baylor experience).” Ann Surg Oncol 7(8): 588-92.

Lee, E. R., et al., Detailed analysis of structures and formulations of cationic lipids for efficient gene transfer to the lung. Hum Gene Ther, 1996. 7(14): p. 1701-1717.

Lee, S. G., et al., Enhancement of adenoviral transduction with polycationic liposomes in vivo. Cancer Gene Ther, 2000. 7(10): p. 1329-1335.

Ludtke, S. J., P. R. Baldwin, and W. Chiu, EMAN: semiautomated software for high-resolution single-particle reconstructions. J Struct Biol, 1999. 128(1): p. 82-97.

McGough, A., et al., Cofilin changes the twist of F-actin: implications for actin filament dynamics and cellular function. J Cell Biol, 1997. 138(4): p. 771-781.

Meunier-Durmort, C., et al., Mechanism of adenovirus improvement of cationic liposome-mediated gene transfer. Biochim Biophys Acta, 1997. 1330(1): p. 8-16.

Mirchandani, D., J. Zheng, et al. (1995). “Heterogeneity in intratumor distribution of p53 mutations in human prostate cancer.” Am J Pathol 147(1): 92-101.

Morral, N., et al., Immune responses to reporter proteins and high viral dose limit duration of expression with adenoviral vectors: comparison of E2a wild type and E2a deleted vectors. Hum Gene Ther, 1997. 8(10): p. 1275-1286.

Mulvihill, S., R. Warren, et al. (2001). “Safety and feasibility of injection with an E1B-55 kDa gene-deleted, replication-selective adenovirus (ONYX-015) into primary carcinomas of the pancreas: a phase I trial.” Gene Ther 8(4): 308-15.

Natsume, A., et al., Cationic liposome conjugation to recombinant adenoviral vector reduces viral antigenicity. Jpn J Cancer Res, 2000. 91(4): p. 363-367.

Nemunaitis, J., C. Cunningham, et al. (2001). “Intravenous infusion of a replication-selective adenovirus (ONYX-015) in cancer patients: safety, feasibility and biological activity.” Gene Ther 8(10): 746-59.

Nemunaitis, J., I. Ganly, et al. (2000). “Selective replication and oncolysis in p53 mutant tumors with ONYX-015, an E1B-55 kD gene-deleted adenovirus, in patients with advanced head and neck cancer: a phase II trial.” Cancer Res 60(22): 6359-66.

Nemunaitis, J., F. Khuri, et al. (2001). “Phase II trial of intratumoral administration of ONYX-015, a replication-selective adenovirus, in patients with refractory head and neck cancer.” J Clin Oncol 19(2): 289-98.

Nicolau and Sene, “Liposome-mediated DNA transfer in eukaryotic cells: dependence of the transfer efficiency upon the type of liposomes used and the host cell cycle stage,” Biochem. Biophys. Acta, 721:185-190, 1982.

Nicolau et al., “Liposomes as carriers for in vivo gene transfer and expression,” Methods Enzymol., 149:157-176, 1987.

Nunes, F. A., et al., Gene transfer into the liver of nonhuman primates with E1-deleted recombinant adenoviral vectors: safety of readministration. Hum Gene Ther, 1999. 10(15): p. 2515-2526.

O'Neal, W. K., et al., Toxicity associated with repeated administration of first-generation adenovirus vectors does not occur with a helper-dependent vector. Mol Med, 2000. 6(3): p. 179-195.

Parks, R., C. Evelegh, and F. Graham, Use of helper-dependent adenoviral vectors of alternative serotypes permits repeat vector administration. Gene Ther, 1999. 6(9): p. 1565-1573.

Perales et al., “Gene transfer in vivo: sustained expression and regulation of genes introduced into the liver by receptor-targeted uptake,” Proc. Natl. Acad. Sci. USA, 91:4086-4090, 1994.

Quantin, B., et al., Adenovirus as an expression vector in muscle cells in vivo. Proc Natl Acad Sci USA, 1992. 89(7): p. 2581-2584.

Qiu, C., et al., Cationic liposomes enhance adenovirus entry via a pathway independent of the fiber receptor and alpha(v)-integrins. Hum Gene Ther, 1998. 9(4): p. 507-520.

Rothmann, T., A. Hengstermann, et al. (1998). “Replication of ONYX-015, a potential anticancer adenovirus, is independent of p53 status in tumor cells.” J Virol 72(12): 9470-8.

Ruben et al., “Covalently closed circles of adenovirus 5 DNA”, Nature 301: 172-174.

Scheule, R. K., et al., Basis of pulmonary toxicity associated with cationic lipid-mediated gene transfer to the mammalian lung. Hum Gene Ther, 1997. 8(6): p. 689-707.

Seth, P., et al., Mechanism of enhancement of DNA expression consequent to cointemalization of a replication-deficient adenovirus and unmodified plasmid DNA. J Virol, 1994. 68(2): p. 933-940.

Sung, M. -W. et al., “Cationic liposome-enhanced adenoviral gene transfer in a murine head and neck cancer model,” Anticancer Research, 20:1653-1656, 2000.

Szoka and Papahadjopoulos, “Procedure for Preparation of Liposomes With Large Internal Aqueous Space . . . ”, Proc. Natl. Acad. Sci., 75:4194-4198, 1978.

Templeton, N. S., et al., Improved DNA: liposome complexes for increased systemic delivery and gene expression. Nat Biotechnol, 1997. 15(7): p. 647-652.

Templeton, N. S. Myths concerning the use of cationic liposomes in vivo. Expert Opin. Biol. Ther. 3(l):1-13, 2003.

Toogood, C. I., J. Crompton, and R. T. Hay, Antipeptide antisera define neutralizing epitopes on the adenovirus hexon. J Gen Virol, 1992. 73(Pt 6): p. 1429-1435.

Turnell, A. S., R. J. Grand, et al. (1999). “The replicative capacities of large E1B-null group A and group C adenoviruses are independent of host cell p53 status.” J Virol 73(3): 2074-83.

Tursz, T., et al., Phase I study of a recombinant adenovirus-mediated gene transfer in lung cancer patients. J Natl Cancer Inst, 1996. 88(24): p. 1857-1863.

Vollmer, C. M., A. Ribas, et al. (1999). “p53 selective and nonselective replication of an E1B-deleted adenovirus in hepatocellular carcinoma.” Cancer Res 59(17): 4369-74.

Wagner et al., Science, 260:1510-1513, 1990.

Welsh, M. J., et al., Cystic fibrosis gene therapy using an adenovirus vector: in vivo safety and efficacy in nasal epithelium. Hum Gene Ther, 1994. 5(2): p. 209-219.

Wilson, J. M., Cystic fibrosis. Vehicles for gene therapy. Nature, 1993. 365(6448): p. 691-692.

Wohlfart, C., Neutralization of adenoviruses: kinetics, stoichiometry, and mechanisms. J Virol, 1988. 62(7): p. 2321-2328.

Wong et al., “Appearance of beta-lactamase activity in animal cells upon liposome-mediated gene transfer,” Gene., 10(2):87-94, 1980.

Wu and Wu, “Receptor-mediated in vitro gene transfections by a soluble DNA carrier system,” J. Biol. Chem., 262:4429-4432, 1987.

Wu and Wu, Adv. Drug Delivery Rev., 12:159-167, 1993.

Yang, Y., G. Trinchieri, and J. M. Wilson, Recombinant IL-12 prevents formation of blocking IgA antibodies to recombinant adenovirus and allows repeated gene therapy to mouse lung. Nat Med, 1995. 1(9): p. 890-893.

Yang, Y., et al., Cellular and humoral immune responses to viral antigens create barriers to lung-directed gene therapy with recombinant adenoviruses. J Virol, 1995. 69(4): p. 2004-2015.

Yotnda, P., H. Onishi, H E. Heslop, D M. Shayakhmetov, A. Lieber, M K. Brenner, and. A. Davis. Highly efficient transduction of primitive hematopoietic stem cells by modified Adenovirus. Hum Gene Ther. Apr. 10, 2001;12(6):659-70.

Yotnda, P., T. Dong-Hua Chen, W. Chiu, P. A. Piedra, A. Davis, N. Smyth Templeton, M. K. Brenner. Bilamellar Cationic Liposomes Protect Adenovectors from Pre-existing Humoral Immune Responses. Molecular Therapy. Mar. 1, 2002: 5(3). 233-241.

Zabner, J., et al., Cellular and molecular barriers to gene transfer by a cationic lipid. J Biol Chem, 1995. 270(32): p. 18997-19007.

Zhang, Y., et al., Acute cytokine response to systemic adenoviral vectors in mice is mediated by dendritic cells and macrophages. Mol Ther, 2001. 3(5 Pt 1): p. 697-707.

Zsengeller, Z., et al., Internalization of adenovirus by alveolar macrophages initiates early proinflammatory signaling during acute respiratory tract infection. J Virol, 2000. 74(20): p. 9655-9667.

Zuckerman, J. B., et al., A phase I study of adenovirus-mediated transfer of the human cystic fibrosis transmembrane conductance regulator gene to a lung segment of individuals with cystic fibrosis. Hum Gene Ther, 1999. 10(18): p. 2973-2985.

One skilled in the art readily appreciates that the present invention is well adapted to carry out the objectives and obtain the ends and advantages mentioned as well as those inherent therein. Particles, compositions, treatments, methods, kits, procedures and techniques described herein are presently representative of the preferred embodiments and are intended to be exemplary and are not intended as limitations of the scope. Changes therein and other uses will occur to those skilled in the art which are encompassed within the spirit of the invention or defined by the scope of the claims appended herein.

We claim:

1. As a composition of matter, a liposome comprising:
adenoviral DNA, wherein the adenoviral DNA comprises a circular form; and
a therapeutic polynucleotide.

2. The composition of claim 1, wherein the adenoviral DNA comprises the therapeutic polynucleotide.

3. The composition of claim 1, wherein the adenoviral DNA is contained within the liposome.

4. The composition of claim 1, wherein the adenoviral DNA is housed within an adenoviral particle.

5. The composition of claim 1, wherein the adenoviral DNA is native adenoviral DNA.

6. The composition of claim 1, wherein the adenoviral DNA is recombinant.

7. The composition of claim 1, wherein the adenoviral DNA is replication-deficient.

8. The composition of claim 1, wherein the adenoviral DNA is dl1520.

9. The composition of claim 1, wherein the liposome is a bilammelar liposome.

10. The composition of claim 1, wherein the liposome is comprised of DOTAP.

11. The composition of claim 1, wherein the liposome is comprised of extruded DOTAP-cholesterol.

12. The composition of claim 1, wherein said composition further comprises humoral immune response-neutralizing activity.

13. A therapeutic composition, comprising:
a liposome, comprising:
adenoviral DNA, wherein the adenoviral DNA comprises a circular form; and
a therapeutic polynucleotide; and
a pharmaceutical carrier.

14. The composition of claim 13, wherein the adenoviral DNA comprises the therapeutic polynucleotide.

15. The composition of claim 13, wherein the adenoviral DNA is contained within the liposome.

16. The composition of claim 13, wherein the adenoviral DNA is native adenoviral DNA.

17. The composition of claim 13, wherein the adenoviral DNA is recombinant.

18. The composition of claim 13, wherein the adenoviral DNA is replication-deficient.

19. The composition of claim 13, wherein the adenoviral DNA is dl1520.

20. The composition of claim 13, wherein the liposome is a bilammelar liposome.

21. The composition of claim 13, wherein the liposome is comprised of DOTAP.

22. The composition of claim 13, wherein the liposome is comprised of extruded DOTAP-cholesterol.

23. The composition of claim 13, wherein said composition further comprises humoral immune response-neutralizing activity.

24. A vaccine comprising the composition of claim 13.

25. A method of treating a disease in an individual comprising the step of administering to the individual a composition comprising a liposome, said liposome comprising:
adenoviral DNA, wherein the adenoviral DNA comprises a circular form;
a therapeutic polynucleotide; and
a pharmaceutical carrier.

26. The method of claim 25, wherein the adenoviral DNA comprises the therapeutic polynucleotide.

27. The method of claim 25, wherein the adenoviral DNA is contained within the liposome.

28. The method of claim 25, wherein the composition is administered at least a second time.

29. The method of claim 25, wherein the individual is a warm-blooded animal.

30. The method of claim 29, wherein the animal is a human.

31. The method of claim 25, wherein the disease is cancer.

32. The method of claim 31, wherein the cancer is lung cancer.

33. The method of claim 25, wherein the therapeutic polynucleotide encodes p53, BRCA1, BRCA2, a bone morphogenetic protein, an interleukin, thymidine kinase, or cytosine deaminase.

34. The method of claim 25, wherein the adenoviral DNA is dl1520.

35. The method of claim 25, wherein the liposome is a bilammelar liposome.

36. The method of claim 25, wherein the liposome is comprised of DOTAP.

37. The method of claim 25, wherein the liposome is comprised of extruded DOTAP-cholesterol.

38. The method of claim 25, wherein said composition further comprises humoral immune response-neutralizing activity.

39. A method of preventing a disease in an individual comprising the step of administering to the individual a composition comprising a liposome, said liposome comprising:
adenoviral DNA, wherein the adenoviral DNA comprises a circular form;
a therapeutic polynucleotide; and
a pharmaceutical carrier.

40. The method of claim 39, wherein the composition is administered at least a second time.

41. As a composition of matter, a liposome comprising within a circular adenoviral DNA, wherein the adenoviral DNA comprises a therapeutic polynucleotide.

42. As a composition of matter, a liposome comprised of DOTAP, said liposome comprising:
adenoviral DNA, wherein the adenoviral DNA comprises a linear form; and
a therapeutic polynucleotide.

43. The composition of claim 42, wherein said adenoviral DNA comprises said therapeutic polynucleotide.

44. The composition of claim 42, wherein the adenoviral DNA is comprised within the liposome.

45. The composition of claim 42, wherein the adenoviral DNA is not housed within an adenoviral particle.

46. A mixture of liposomes and adenoviral DNA, wherein the adenoviral DNA is housed within the liposomes, said mixture substantially lacking adenoviral DNA outside said liposomes.

47. The mixture of claim 46, wherein the adenoviral DNA comprises a therapeutic polynucleotide.

48. The mixture of claim 46, wherein the adenoviral DNA is not housed within an adenoviral particle.
